(19) United States
(12) Patent Application Publication
(10) Pub. No.: US 2026/0064344 A1
(43) Pub. Date: Mar. 5, 2026

(54) SYSTEMS AND METHODS OF TRAVERSING WEBPAGES USING AN ARTIFICIAL INTELLIGENCE AGENT AND PERFORMING ACTIONS BASED ON COLLECTED INFORMATION

(71) Applicant: Meta Platforms Technologies, LLC, Menlo Park, CA (US)

(72) Inventors: Nicholas Wallen, Atlanta, GA (US); Benjamin Neal Bethurum, Shoreline, WA (US); Willy Huang, San Francisco, CA (US); Sean Garrett Kelly, Fall City, WA (US); Hayden Schoen, Evanston, IL (US); Shengzhi Wu, Belmont, CA (US)

(21) Appl. No.: 19/382,803

(22) Filed: Nov. 7, 2025

Related U.S. Application Data
(63) Continuation of application No. 19/324,058, filed on Sep. 9, 2025.
Application No. 19/324,058 is a continuation-in-part of application No. 19/209,723, filed on May 15, 2025.
(60) Provisional application No. 63/649,289, filed on May 17, 2024.
(60) Provisional application No. 63/649,907, filed on May 20, 2024.

(51) Int. Cl. G06F 3/14; A61B 5/00; A61B 5/11; G06F 3/01

(52) U.S. Cl. CPC G06F 3/14; A61B 5/1118; A61B 5/6803; A61B 5/7264; G06F 3/011

(57) ABSTRACT

Systems and methods of traversing webpages using an artificial intelligence (AI) agent are disclosed. An example method includes, in response to a user request, received via a wearable device worn by a user, to perform a web-based action, invoking the AI agent. The method includes, causing the AI agent to traverse webpages based on the user request. The AI agent obtains, for each webpage of the webpages, information for performing the web-based action. The method includes generating, by the AI agent, an AI response based on the information, and presenting, at the wearable device, the AI response. The AI response includes recommendations for performing the web-based action. The method further includes presenting, at the wearable device, the AI response; and, in response to user selection, via the wearable device, of a recommendation included in the AI response, causing the AI agent to perform the web-based action based on the recommendation.

### RELATED APPLICATION

This application is a continuation of U.S. patent application Ser. No. 19/324,058, filed Sep. 9, 2025, entitled “Systems And Methods Of Using Artificial Intelligence To Generate Personalized Physical Activity Notifications Based On Data Captured By A Wearable Device,” which is a continuation-in-part of U.S. patent application Ser. No. 19/209,723, filed May 15, 2025, entitled “Wearable Devices Including Artificially Intelligent Systems For Generating And Presenting Guidance To Wearers,” which claims priority to U.S. Provisional Application Ser. No. 63/649,289, filed May 17, 2024, entitled “Methods Of Interacting With Wearable Devices As A Result Of Artificial Intelligence Determinations, Devices, And Systems Thereof,” and U.S. Provisional Application Ser. No. 63/649,907, filed May 20, 2024, entitled “Artificial-Intelligence-Assisted Activity Management And Interaction Assistance For Use With Smart Glasses, And Devices, Systems, And Methods Thereof,” each of which is incorporated herein by reference.

### TECHNICAL FIELD

This relates generally to approaches for interacting with an artificially intelligent agent and, more specifically, utilizing artificially intelligent agent included at wearable devices to augment user experiences.

### BACKGROUND

While artificial intelligence is used in different manners, commercial AI is usually only accessible in inconvenient manners, such as interacting with an artificial intelligence on a website or receiving AI generated content in relation to an internet search. These examples have drawbacks as it limits the user's experience with AI generated content to very siloed experiences and also has a high burden on the user for accessing/interacting with the AI.

As such, there is a need to address one or more of the above-identified challenges. A brief summary of solutions to the issues noted above are described below.

### SUMMARY

In one example embodiment, a wearable device for generating orchestrated guidance based on an activity of a user is described herein. The example wearable device can be a head-wearable device including a display, one or more sensors, and one or more programs. The one or more programs are stored in memory and configured to be executed by one or more processors, the one or more programs including instructions for, in response to an indication that an artificial intelligence (AI) agent trigger condition is present, providing an AI agent sensor data obtained by the wearable device. The one or more programs include instructions for determining, by the AI agent, a context-based activity based on the sensor data obtained by the wearable device, and generating, by the AI agent, orchestrated guidance based on the context-based activity. The orchestrated guidance includes a recommended action for performing the context-based activity. The one or more programs further include instructions for presenting the orchestrated guidance at the wearable device.

In another example embodiment, a method for generating orchestrated guidance based on an activity of a user is described herein. The method can be performed by a head-wearable device including a display and one or more sensors. The method includes, in response to an indication that an artificial intelligence (AI) agent trigger condition is present, providing an AI agent sensor data obtained by the head-wearable device. The method also includes determining, by the AI agent, a context-based activity based on the sensor data obtained by the wearable device, and generating, by the AI agent, orchestrated guidance based on the context-based activity. The orchestrated guidance includes a recommended action for performing the context-based activity. The method further includes presenting the orchestrated guidance at the wearable device.

In yet another example embodiment, a non-transitory, computer-readable storage medium including executable instructions that, when executed by one or more processors of a wearable device (e.g., a head-wearable device), cause the one or more processors to generate orchestrated guidance based on an activity of a user is described herein. The executable instructions, when executed by one or more processors, cause the one or more processors to, in response to an indication that an artificial intelligence (AI) agent trigger condition is present, provide an AI agent sensor data obtained by the head-wearable device. The executable instructions, when executed by one or more processors, cause the one or more processors to determine, by the AI agent, a context-based activity based on the sensor data obtained by the wearable device, and generate, by the AI agent, orchestrated guidance based on the context-based activity. The orchestrated guidance includes a recommended action for performing the context-based activity. The executable instructions, when executed by one or more processors, cause the one or more processors to present the orchestrated guidance at the wearable device.

In one example embodiment, a wearable device for facilitating performance of a physical activity performed by user is described herein. The example wearable device can be a head-wearable device including a display, one or more sensors, and one or more programs. The one or more programs are stored in memory and configured to be executed by one or more processors, the one or more programs including instructions for, in response to an indication that a user of a head-wearable device is participating in an activity, obtaining data associated with an on-going activity performed by the user of the head-wearable device. The one or more programs include instructions for generating, by an artificial intelligence (AI) agent, a context-based response based, in part, on the data associated with the on-going activity performed by the user of the head-wearable device. The one or more programs include instructions for presenting, at the head-wearable device, context-based response. The context-based response is presented within a portion of a field of view of the user.

In another example embodiment, a method for facilitating performance of a physical activity performed by user is described herein. The method includes, in response to an indication that a user of a head-wearable device is participating in an activity, obtaining data associated with an on-going activity performed by the user of the head-wearable device. The method also includes generating, by an artificial intelligence (AI) agent, a context-based response based, in part, on the data associated with the on-going activity performed by the user of the head-wearable device. The method further includes presenting, at the head-wearable device, context-based response, wherein the context-based response is presented within a portion of a field of view of the user.

In yet another example embodiment, a non-transitory, computer-readable storage medium including executable instructions that, when executed by one or more processors of a wearable device (e.g., a head-wearable device), cause the one or more processors to facilitate performance of a physical activity performed by user is described herein. The executable instructions, when executed by one or more processors, cause the one or more processors to, in response to an indication that a user of a head-wearable device is participating in an activity, obtain data associated with an on-going activity performed by the user of the head-wearable device. The executable instructions, when executed by one or more processors, cause the one or more processors to generate, by an artificial intelligence (AI) agent, a context-based response based, in part, on the data associated with the on-going activity performed by the user of the head-wearable device. The executable instructions, when executed by one or more processors, cause the one or more processors to present, at the head-wearable device, context-based response, wherein the context-based response is presented within a portion of a field of view of the user.

Instructions that cause performance of the methods and operations described herein can be stored on a non-transitory computer readable storage medium. The non-transitory computer-readable storage medium can be included on a single electronic device or spread across multiple electronic devices of a system (computing system). A non-exhaustive of list of electronic devices that can either alone or in combination (e.g., a system) perform the method and operations described herein include an extended-reality (XR) headset/glasses (e.g., a mixed-reality (MR) headset or a pair of augmented-reality (AR) glasses as two examples), a wrist-wearable device, an intermediary processing device, a smart textile-based garment, etc. For instance, the instructions can be stored on a pair of AR glasses or can be stored on a combination of a pair of AR glasses and an associated input device (e.g., a wrist-wearable device) such that instructions for causing detection of input operations can be performed at the input device and instructions for causing changes to a displayed user interface in response to those input operations can be performed at the pair of AR glasses. The devices and systems described herein can be configured to be used in conjunction with methods and operations for providing an XR experience. The methods and operations for providing an XR experience can be stored on a non-transitory computer-readable storage medium.

The devices and/or systems described herein can be configured to include instructions that cause the performance of methods and operations associated with the presentation and/or interaction with an extended-reality (XR) headset. These methods and operations can be stored on a non-transitory computer-readable storage medium of a device or a system. It is also noted that the devices and systems described herein can be part of a larger, overarching system that includes multiple devices. A non-exhaustive of list of electronic devices that can, either alone or in combination (e.g., a system), include instructions that cause the performance of methods and operations associated with the presentation and/or interaction with an XR experience include an extended-reality headset (e.g., a mixed-reality (MR) headset or a pair of augmented-reality (AR) glasses as two examples), a wrist-wearable device, an intermediary processing device, a smart textile-based garment, etc. For example, when an XR headset is described, it is understood that the XR headset can be in communication with one or more other devices (e.g., a wrist-wearable device, a server, intermediary processing device) which together can include instructions for performing methods and operations associated with the presentation and/or interaction with an extended-reality system (i.e., the XR headset would be part of a system that includes one or more additional devices). Multiple combinations with different related devices are envisioned, but not recited for brevity.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIGS. 1A-1N illustrate invocation of an artificially intelligent agent at one or more wearable devices for providing guidance based on an activity of a user, in accordance with some embodiments.

FIGS. 2A-2R illustrate context-based responses generated by an artificially intelligent agent based on activities performed by a user, in accordance with some embodiments.

FIGS. 3A-3D illustrate example user interfaces and additional features available at an AI assistive system, in accordance with some embodiments.

FIGS. 4A and 4B illustrate example sequences of user interactions with personalized assistive systems, in accordance with some embodiments.

FIG. 5 illustrates a flow chart of a method for generating orchestrated guidance based on an activity of a user, in accordance with some embodiments.

FIG. 6 illustrates a flow chart of a method for facilitating performance of a physical activity performed by user, in accordance with some embodiments.

FIGS. 7A-7C-2 illustrate example XR systems that include AR and MR systems, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

### Overview

Embodiments of this disclosure can include or be implemented in conjunction with various types of extended-realities (XRs) such as mixed-reality (MR) and augmented-reality (AR) systems. MRs and ARs, as described herein, are any superimposed functionality and/or sensory-detectable presentation provided by MR and AR systems within a user's physical surroundings. Such MRs can include and/or represent virtual realities (VRs) and VRs in which at least some aspects of the surrounding environment are reconstructed within the virtual environment (e.g., displaying virtual reconstructions of physical objects in a physical environment to avoid the user colliding with the physical objects in a surrounding physical environment). In the case of MRs, the surrounding environment that is presented through a display is captured via one or more sensors configured to capture the surrounding environment (e.g., a camera sensor, time-of-flight (ToF) sensor). While a wearer of an MR headset can see the surrounding environment in full detail, they are seeing a reconstruction of the environment reproduced using data from the one or more sensors (i.e., the physical objects are not directly viewed by the user). An MR headset can also forgo displaying reconstructions of objects in the physical environment, thereby providing a user with an entirely VR experience. An AR system, on the other hand, provides an experience in which information is provided, e.g., through the use of a waveguide, in conjunction with the direct viewing of at least some of the surrounding environment through a transparent or semi-transparent waveguide(s) and/or lens(es) of the AR glasses. Throughout this application, the term “extended reality (XR)” is used as a catchall term to cover both ARs and MRS. In addition, this application also uses, at times, a head-wearable device or headset device as a catchall term that covers XR headsets such as AR glasses and MR headsets.

As alluded to above, an MR environment, as described herein, can include, but is not limited to, non-immersive, semi-immersive, and fully immersive VR environments. As also alluded to above, AR environments can include marker-based AR environments, markerless AR environments, location-based AR environments, and projection-based AR environments. The above descriptions are not exhaustive and any other environment that allows for intentional environmental lighting to pass through to the user would fall within the scope of an AR, and any other environment that does not allow for intentional environmental lighting to pass through to the user would fall within the scope of an MR.

The AR and MR content can include video, audio, haptic events, sensory events, or some combination thereof, any of which can be presented in a single channel or in multiple channels (such as stereo video that produces a three-dimensional effect to a viewer). Additionally, AR and MR can also be associated with applications, products, accessories, services, or some combination thereof, which are used, for example, to create content in an AR or MR environment and/or are otherwise used in (e.g., to perform activities in) AR and MR environments.

Interacting with these AR and MR environments described herein can occur using multiple different modalities and the resulting outputs can also occur across multiple different modalities. In one example AR or MR system, a user can perform a swiping in-air hand gesture to cause a song to be skipped by a song-providing application programming interface (API) providing playback at, for example, a home speaker.

A hand gesture, as described herein, can include an in-air gesture, a surface-contact gesture, and or other gestures that can be detected and determined based on movements of a single hand (e.g., a one-handed gesture performed with a user's hand that is detected by one or more sensors of a wearable device (e.g., electromyography (EMG) and/or inertial measurement units (IMUs) of a wrist-wearable device, and/or one or more sensors included in a smart textile wearable device) and/or detected via image data captured by an imaging device of a wearable device (e.g., a camera of a head-wearable device, an external tracking camera setup in the surrounding environment)). “In-air” generally includes gestures in which the user's hand does not contact a surface, object, or portion of an electronic device (e.g., a head-wearable device or other communicatively coupled device, such as the wrist-wearable device), in other words the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single- or double-finger tap on a table, on a user's hand or another finger, on the user's leg, a couch, a steering wheel). The different hand gestures disclosed herein can be detected using image data and/or sensor data (e.g., neuromuscular signals sensed by one or more biopotential sensors (e.g., EMG sensors) or other types of data from other sensors, such as proximity sensors, ToF sensors, sensors of an IMU, capacitive sensors, strain sensors) detected by a wearable device worn by the user and/or other electronic devices in the user's possession (e.g., smartphones, laptops, imaging devices, intermediary devices, and/or other devices described herein).

The input modalities as alluded to above can be varied and are dependent on a user's experience. For example, in an interaction in which a wrist-wearable device is used, a user can provide inputs using in-air or surface-contact gestures that are detected using neuromuscular signal sensors of the wrist-wearable device. In the event that a wrist-wearable device is not used, alternative and entirely interchangeable input modalities can be used instead, such as camera(s) located on the headset/glasses or elsewhere to detect in-air or surface-contact gestures or inputs at an intermediary processing device (e.g., through physical input components (e.g., buttons and trackpads)). These different input modalities can be interchanged based on both desired user experiences, portability, and/or a feature set of the product (e.g., a low-cost product may not include hand-tracking cameras).

While the inputs are varied, the resulting outputs stemming from the inputs are also varied. For example, an in-air gesture input detected by a camera of a head-wearable device can cause an output to occur at a head-wearable device or control another electronic device different from the head-wearable device. In another example, an input detected using data from a neuromuscular signal sensor can also cause an output to occur at a head-wearable device or control another electronic device different from the head-wearable device. While only a couple examples are described above, one skilled in the art would understand that different input modalities are interchangeable along with different output modalities in response to the inputs.

Specific operations described above may occur as a result of specific hardware. The devices described are not limiting and features on these devices can be removed or additional features can be added to these devices. The different devices can include one or more analogous hardware components. For brevity, analogous devices and components are described herein. Any differences in the devices and components are described below in their respective sections.

As described herein, a processor (e.g., a central processing unit (CPU) or microcontroller unit (MCU)), is an electronic component that is responsible for executing instructions and controlling the operation of an electronic device (e.g., a wrist-wearable device, a head-wearable device, a handheld intermediary processing device (HIPD), a smart textile-based garment, or other computer system). There are various types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., VR animations, such as three-dimensional modeling); (iv) a field-programmable gate array (FPGA) that can be programmed and reconfigured after manufacturing and/or customized to perform specific tasks, such as signal processing, cryptography, and machine learning; or (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronic components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs. As described herein, a graphics module is a component or software module that is designed to handle graphical operations and/or processes and can include a hardware module and/or a software module.

As described herein, memory refers to electronic components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, other non-volatile solid state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include (i) profile data, including user account data, user settings, and/or other user data stored by the user; (ii) sensor data detected and/or otherwise obtained by one or more sensors; (iii) media content data including stored image data, audio data, documents, and the like; (iv) application data, which can include data collected and/or otherwise obtained and stored during use of an application; and/or (v) any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronic components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) pogo pins, which may be small, spring-loaded pins configured to provide a charging interface; (v) wireless charging interfaces; (vi) global-positioning system (GPS) interfaces; (vii) Wi-Fi interfaces for providing a connection between a device and a wireless network; and (viii) sensor interfaces.

As described herein, sensors are electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device, such as a simultaneous localization and mapping (SLAM) camera); (ii) biopotential-signal sensors; (iii) IMUs for detecting, for example, angular rate, force, magnetic field, and/or changes in acceleration; (iv) heart rate sensors for measuring a user's heart rate; (v) peripheral oxygen saturation (SpO2) sensors for measuring blood oxygen saturation and/or other biometric data of a user; (vi) capacitive sensors for detecting changes in potential at a portion of a user's body (e.g., a sensor-skin interface) and/or the proximity of other devices or objects; (vii) sensors for detecting some inputs (e.g., capacitive and force sensors); and (viii) light sensors (e.g., ToF sensors, infrared light sensors, or visible light sensors), and/or sensors for sensing data from the user or the user's environment. As described herein biopotential-signal-sensing components are devices used to measure electrical activity within the body (e.g., biopotential-signal sensors). Some types of biopotential-signal sensors include (i) electroencephalography (EEG) sensors configured to measure electrical activity in the brain to diagnose neurological disorders; (ii) electrocardiogramar EKG) sensors configured to measure electrical activity of the heart to diagnose heart problems; (iii) EMG sensors configured to measure the electrical activity of muscles and diagnose neuromuscular disorders; (iv) electrooculography (EOG) sensors configured to measure the electrical activity of eye muscles to detect eye movement and diagnose eye disorders.

As described herein, an application stored in memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications; (x) camera applications; (xi) web-based applications; (xii) health applications; (xiii) AR and MR applications; and/or (xiv) any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). A communication interface can refer to a software layer that enables different software programs to communicate with each other (e.g., APIs and protocols such as HTTP and TCP/IP).

As described herein, a graphics module is a component or software module that is designed to handle graphical operations and/or processes and can include a hardware module and/or a software module.

As described herein, non-transitory computer-readable storage media are physical devices or storage medium that can be used to store electronic data in a non-transitory form (e.g., such that the data is stored permanently until it is intentionally deleted and/or modified).

The systems and methods disclosed herein provide different ways in which wearable devices can utilize artificial intelligence (AI) and/or an AI Agent (also referred to as a AI digital assistant or AI assistant). For example, in some embodiments, a head-wearable device can retrieve information and use the information with an AI agent to generate responses and/or recommendations that are displayed at the head-wearable device and/or another communicatively device. The systems and method disclosed here can be used collaborate with other users (including wearers of other wearable devices), and interact with third party applications using built-in AI models, in accordance with some embodiments. The systems and methods disclosed herein can utilize a user interactable AI agent to perform various tasks at the user's request, as well as utilize the AI agent to monitor situations and provide user-specific assistance.

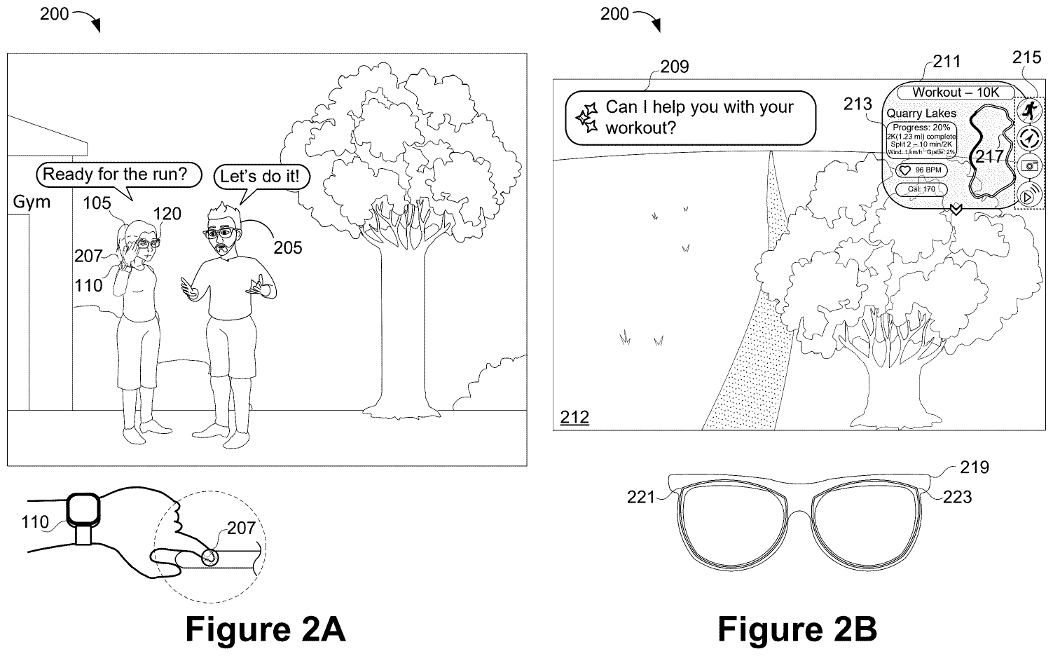

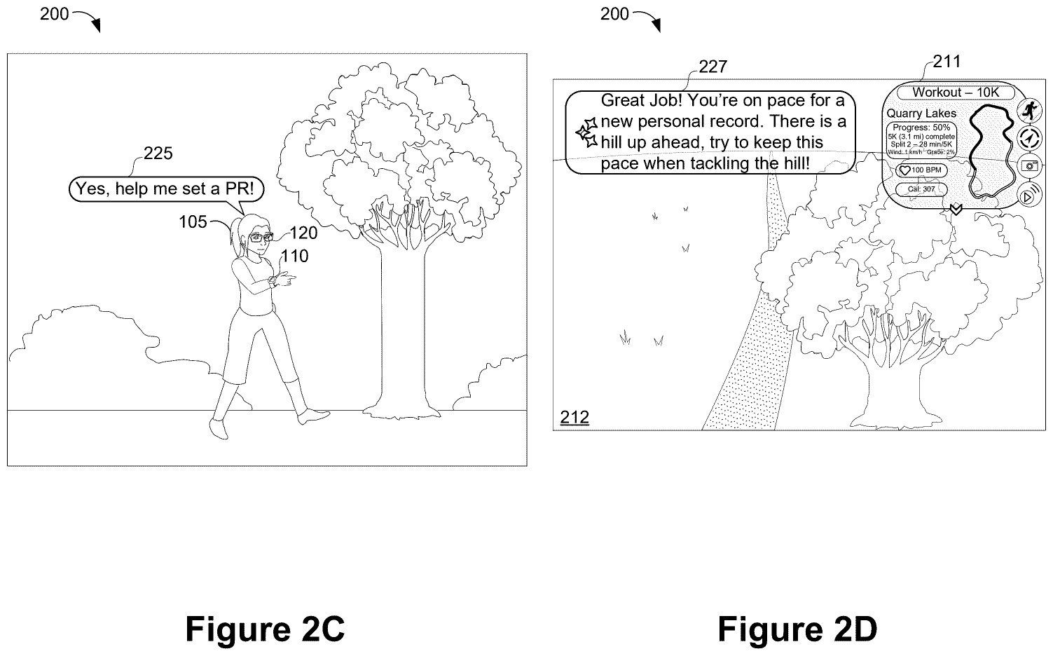

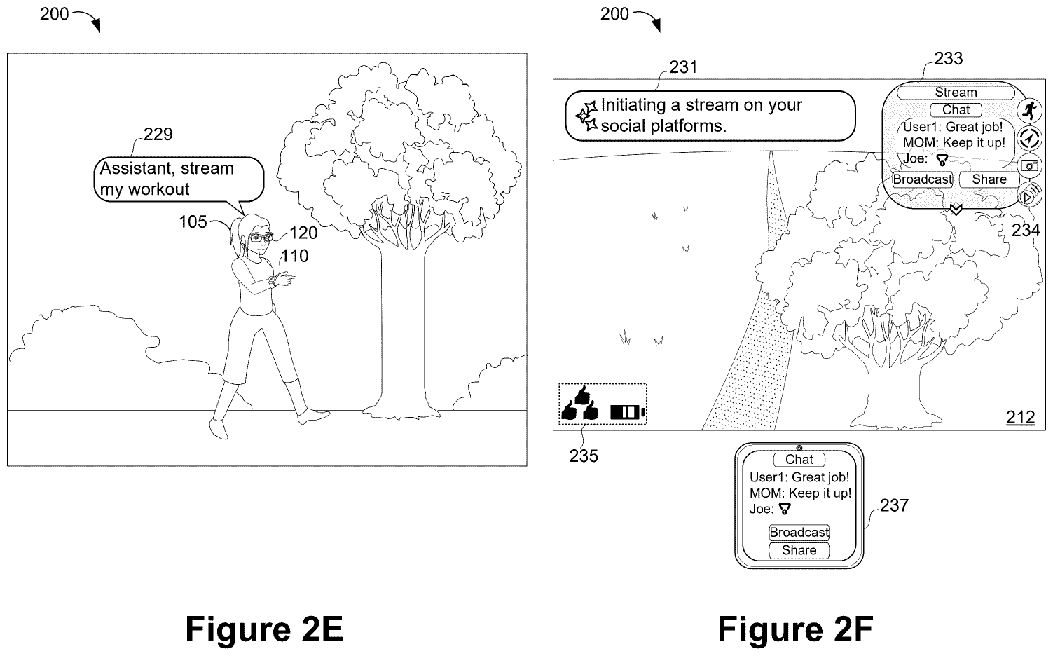

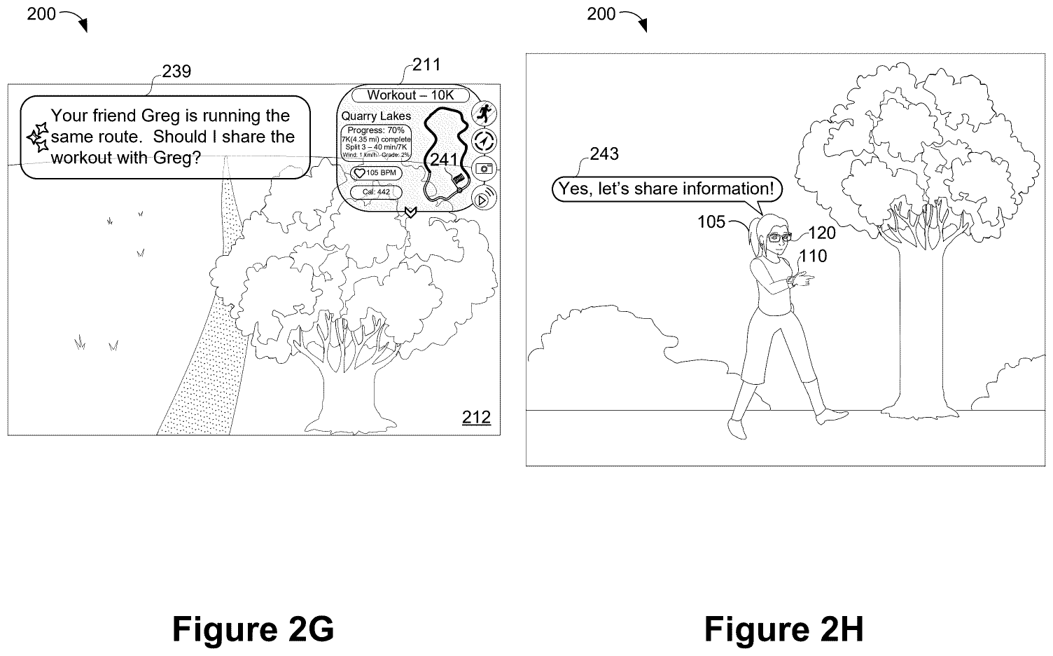

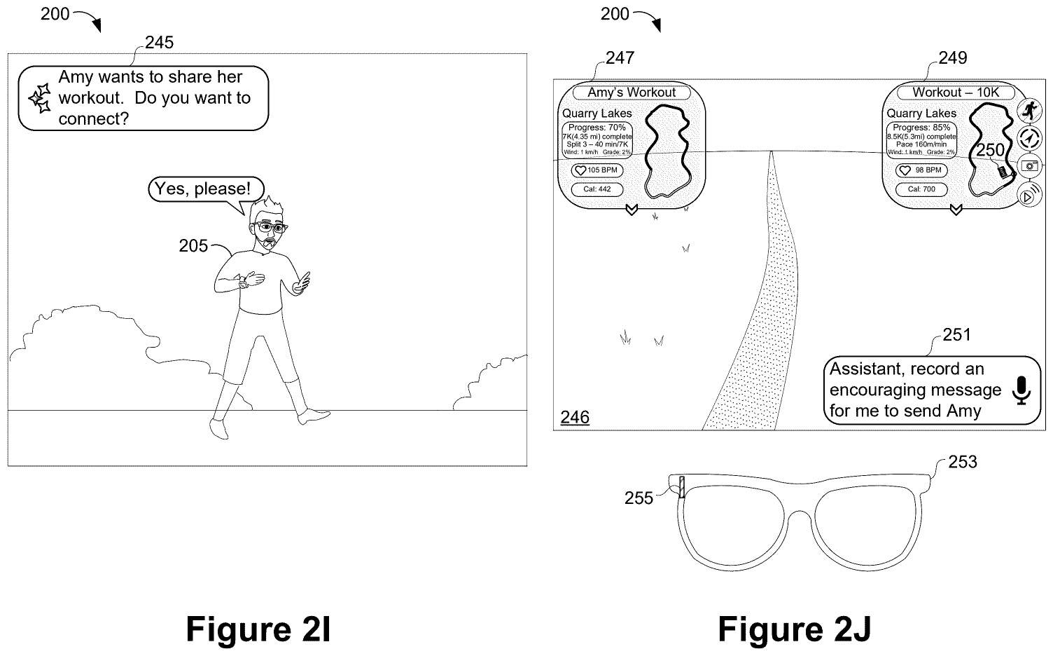

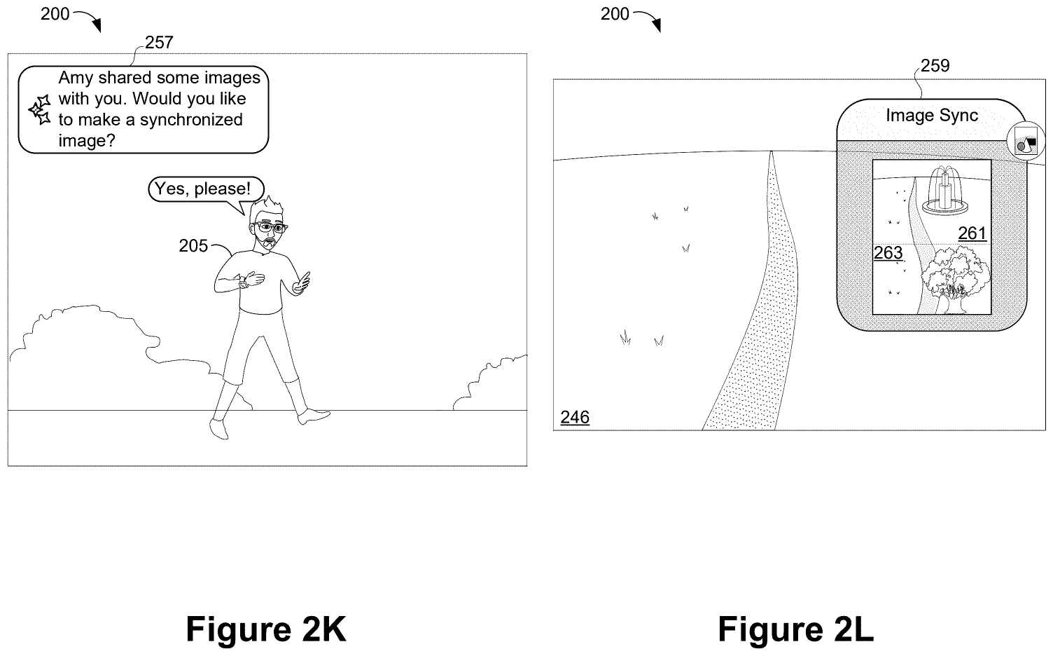

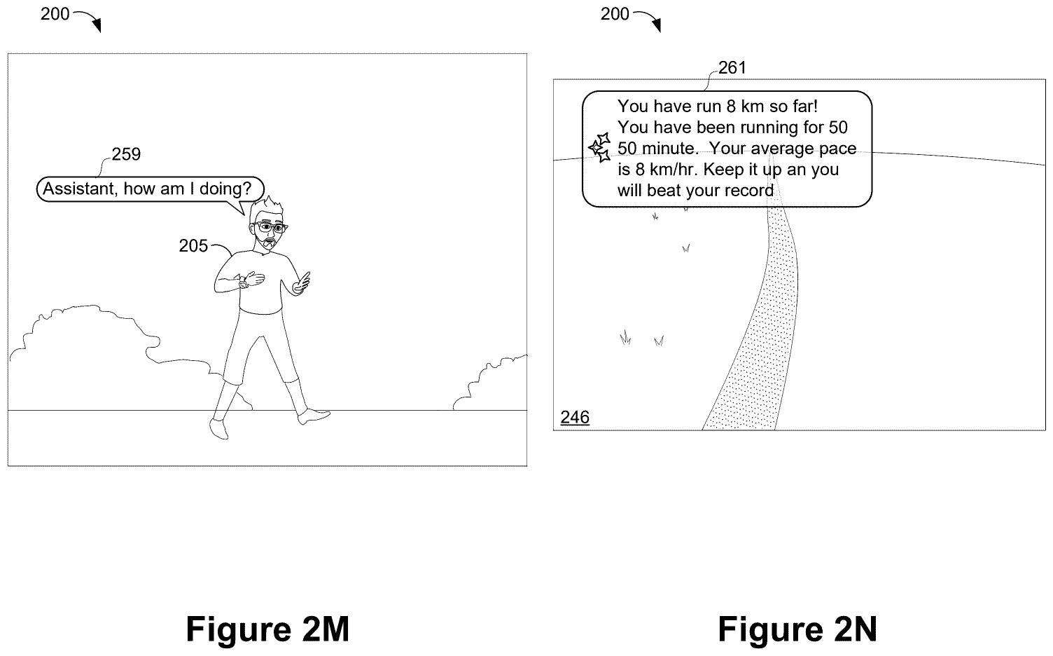

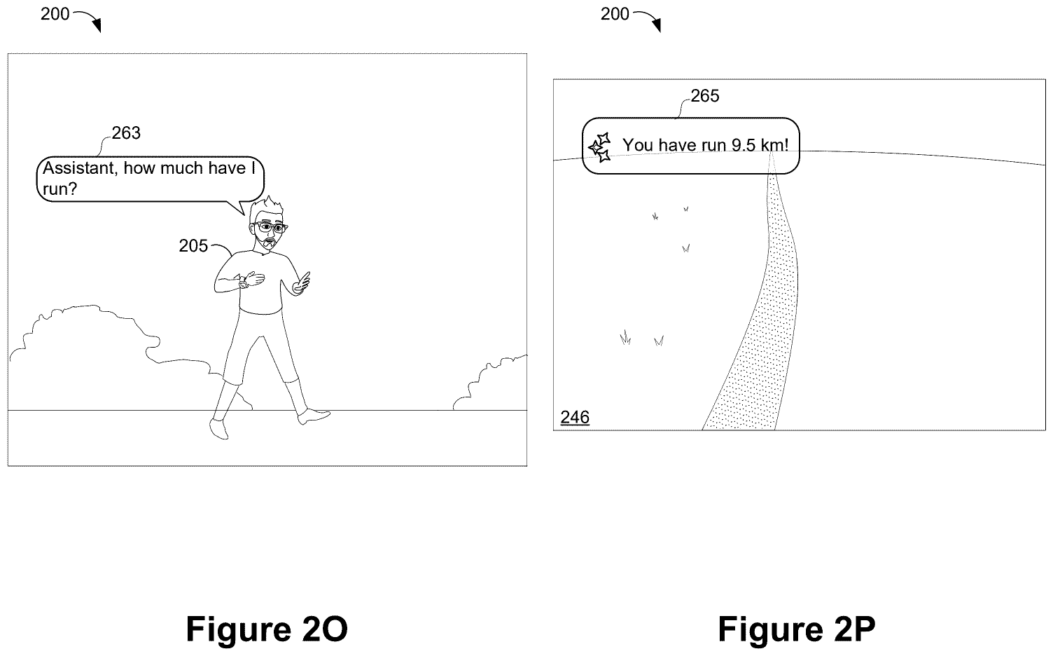

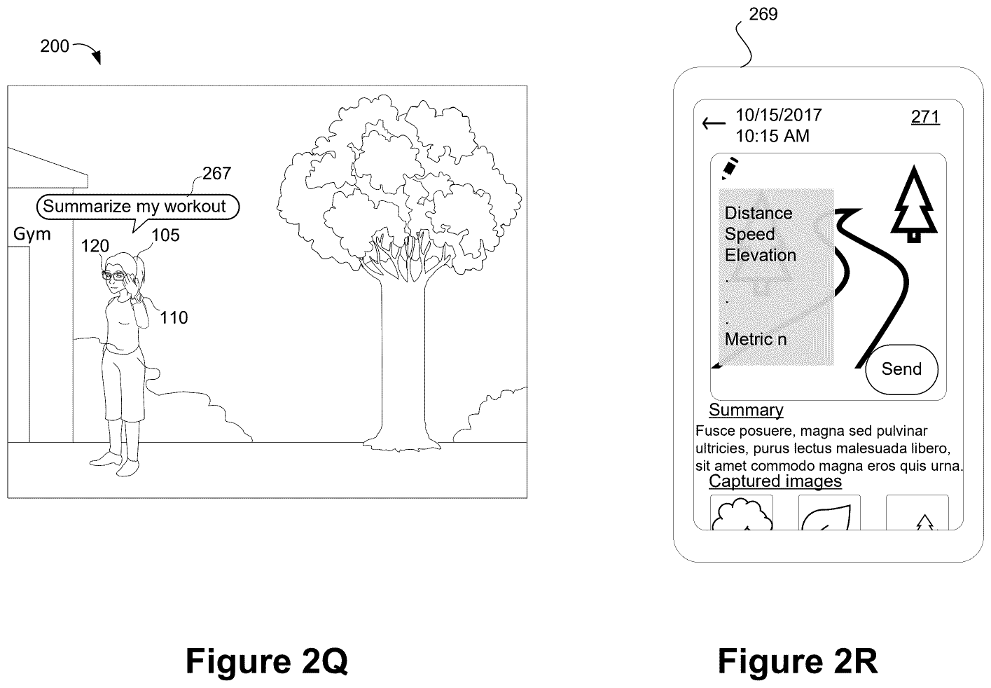

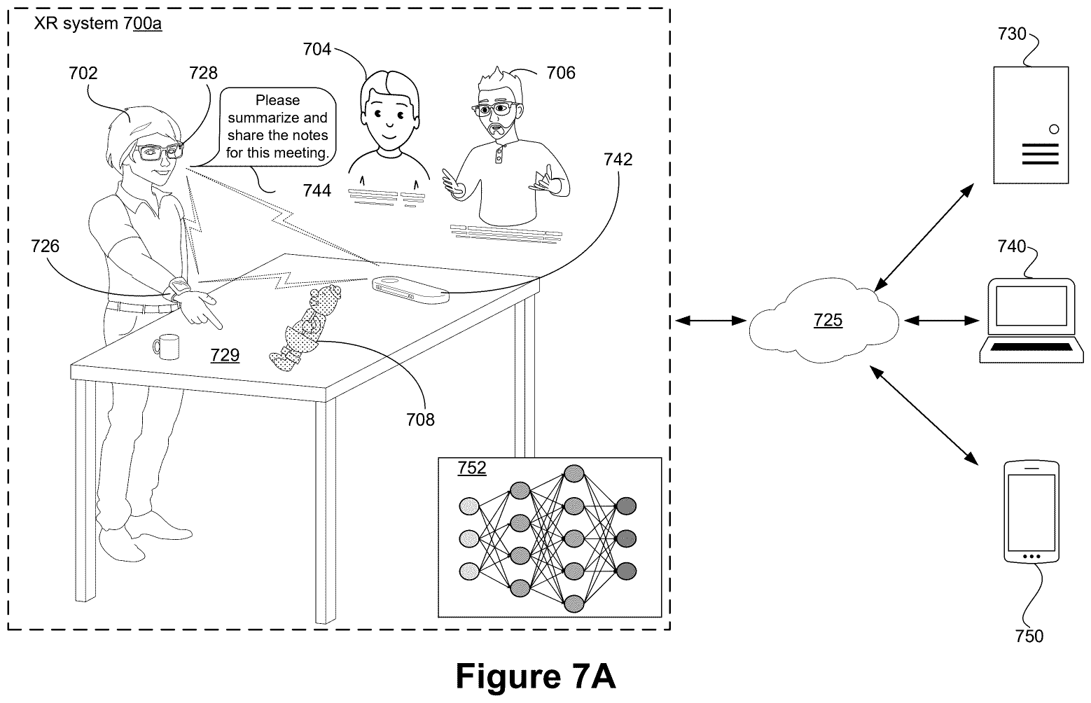

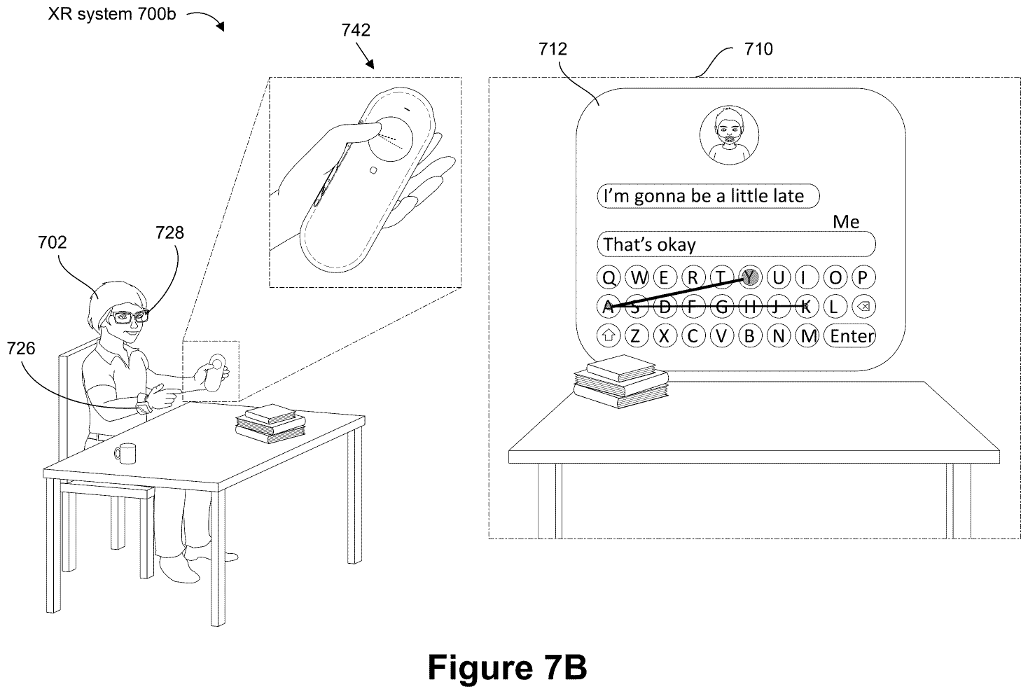

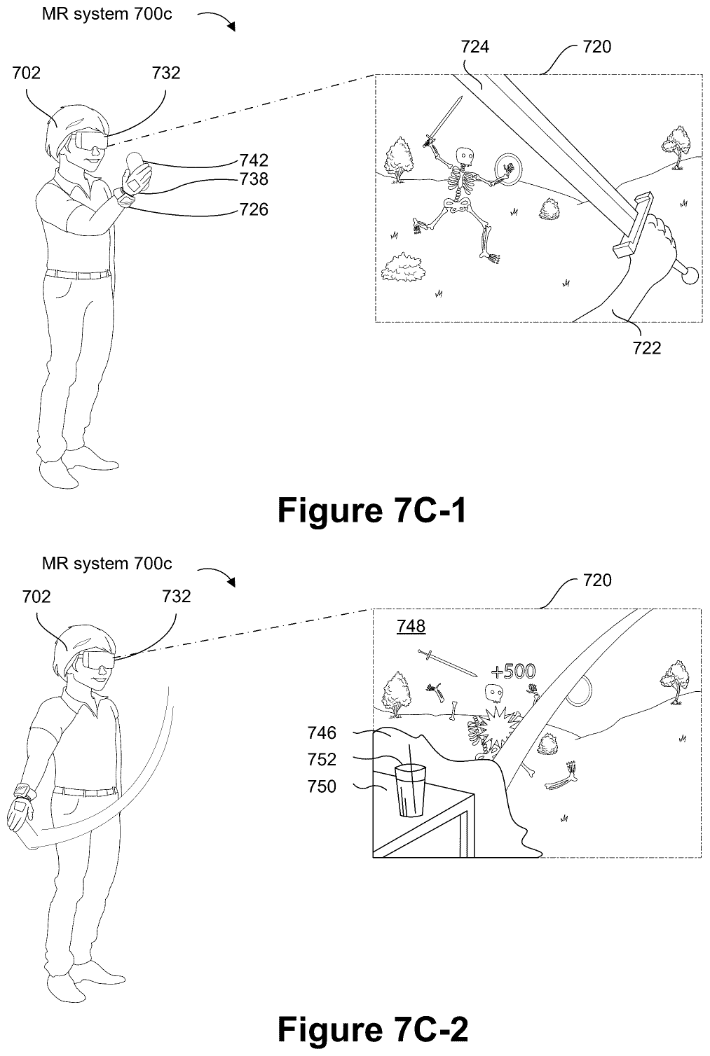

The systems and methods disclosed herein utilize AI agent to work with wearable devices and other devices (e.g., laptop, tablet, watches, desktops, phones, and other internet connected devices) within an ecosystem to accomplish tasks across multiple devices (e.g., XR systems described below in reference to FIGS. 7A-7C-2). For example, an AI agent can be configured to control an aspect of one or more of the other devices based on a request from the user. In some embodiments, the AI agent can also be invoked on different devices based on a determination that the user is interacting with a device other than a wearable device.

In some embodiments, the systems and methods disclosed herein can use an AI agent to augment a user experience. In particular, the AI agent can receive sensor data and/or other information captured by a wearable device, and use the sensor data and/or other information to generate and provide recommended actions and/or context-based responses. For example, a head-wearable device worn by the user can capture information corresponding to a field of view of the user 105 and/or a location of the user to generate and provide recommended actions and/or context-based responses. The systems and methods disclosed herein generate and provide tailored information to a user based on location and/or data received from one or more wearable devices (e.g., sensor data and/or image data of a wrist-wearable device, a head-wearable device, etc.).

The systems and methods disclosed herein utilize an AI agent to collate recorded information (e.g., camera photos and videos) across multiple wearable devices to produce unique media (e.g., a single video which stitches the multiple head-wearable devices video feed into a single viewing experience). In some embodiments, positional data of each communicatively coupled device (e.g., wearable device, such as a head-wearable device) can be used to determine how the media is presented.

The systems and methods disclosed herein utilize an AI agent to work with third-party applications through the use of an API. In other words, the user can use an AI agent implemented at a wearable device to perform a task of applications by utilizing the API to communicate with the AI agent. In some embodiments, the AI agent can be configured to interact with applications and graphical user interfaces (GUIs) without the use of an API.

### Context-Driven Artificially Intelligent Guidance

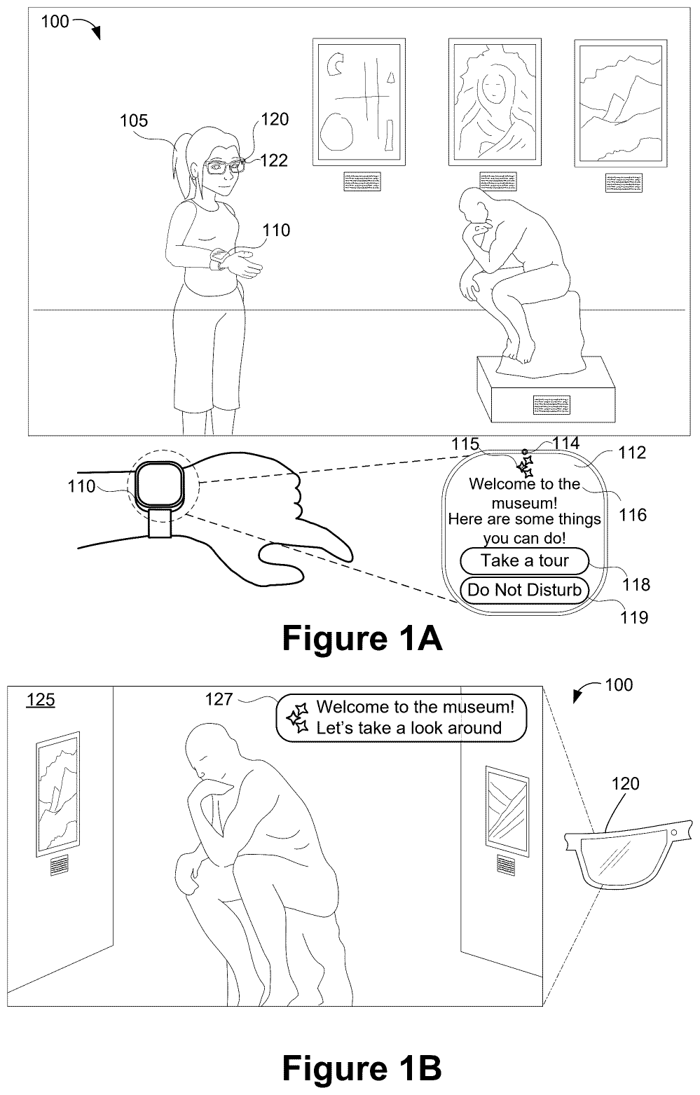

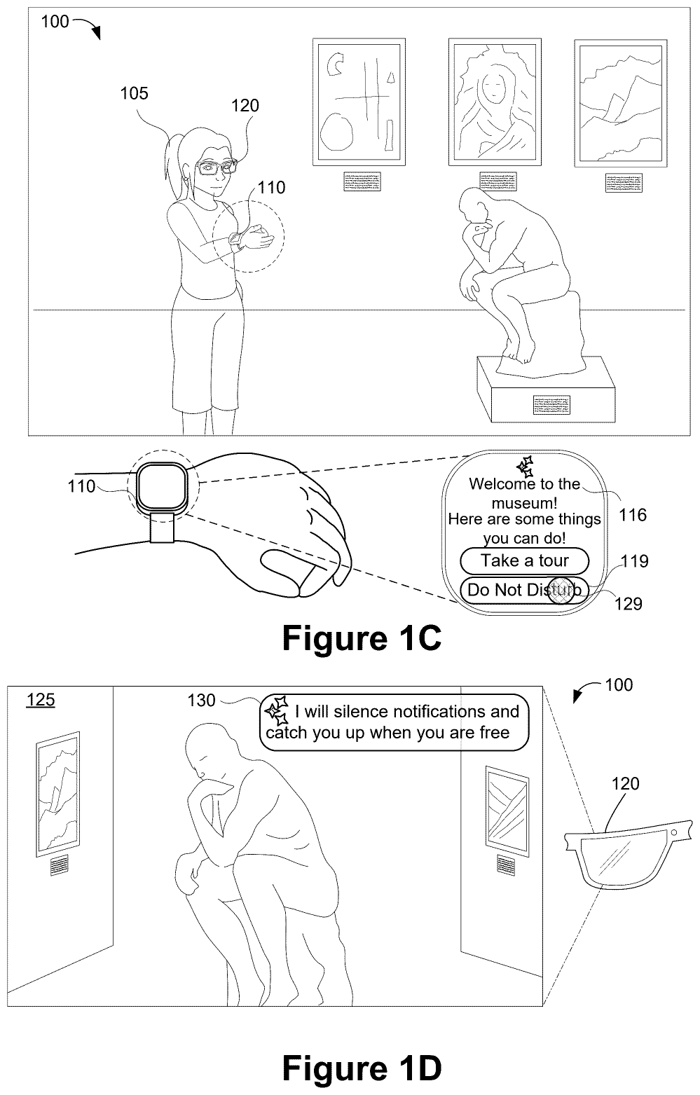

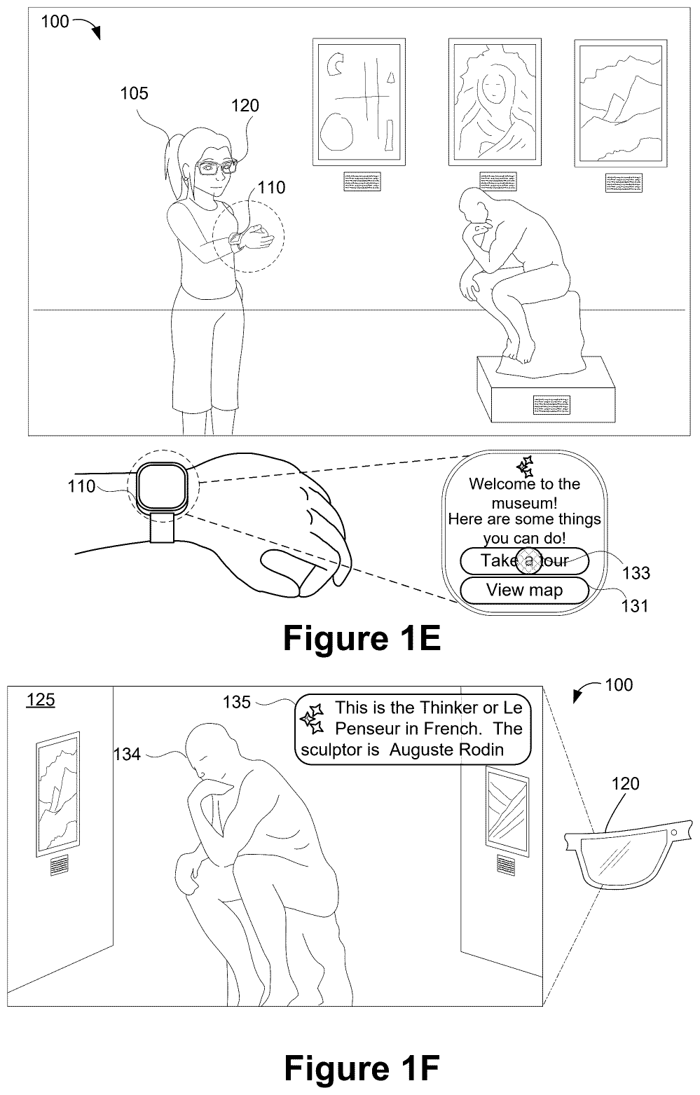

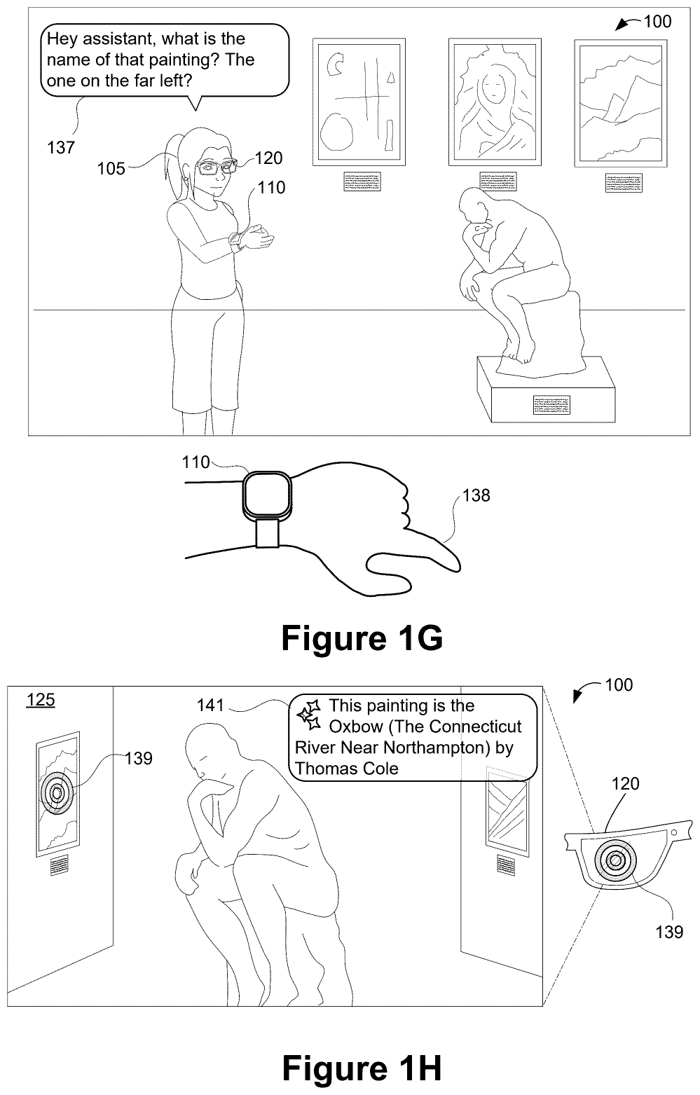

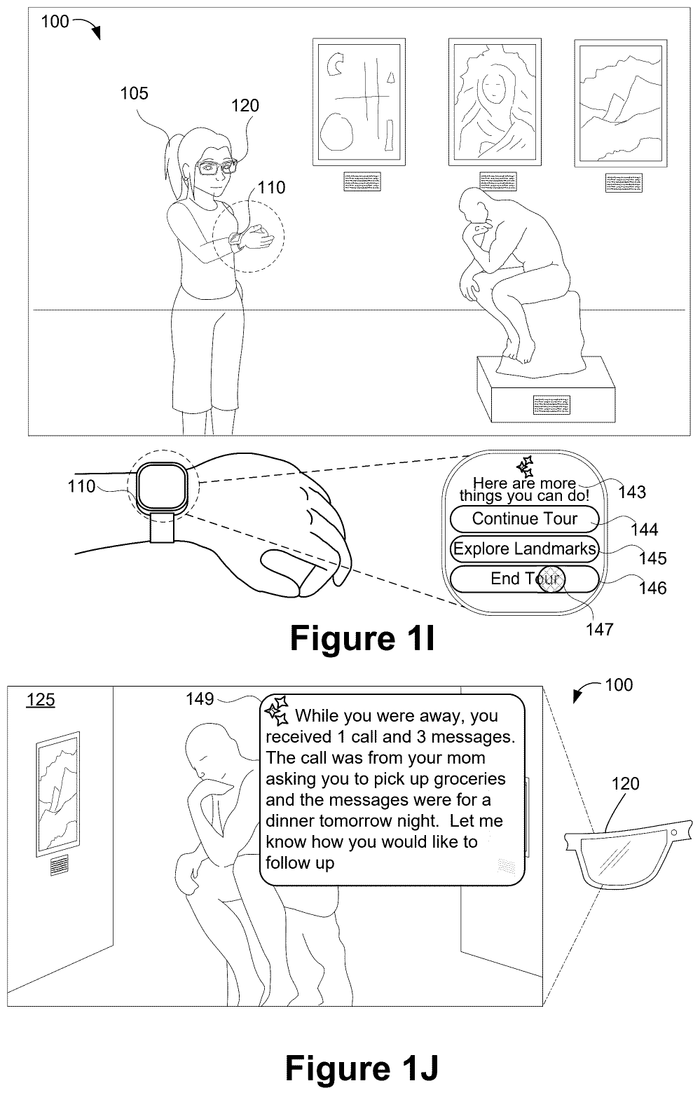

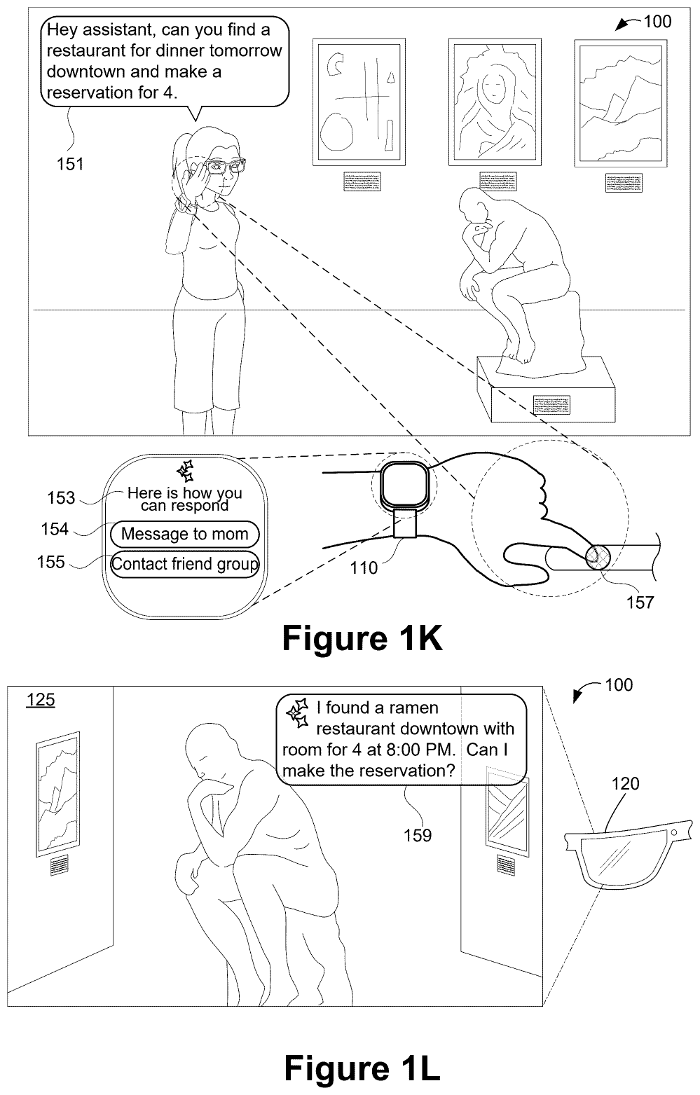

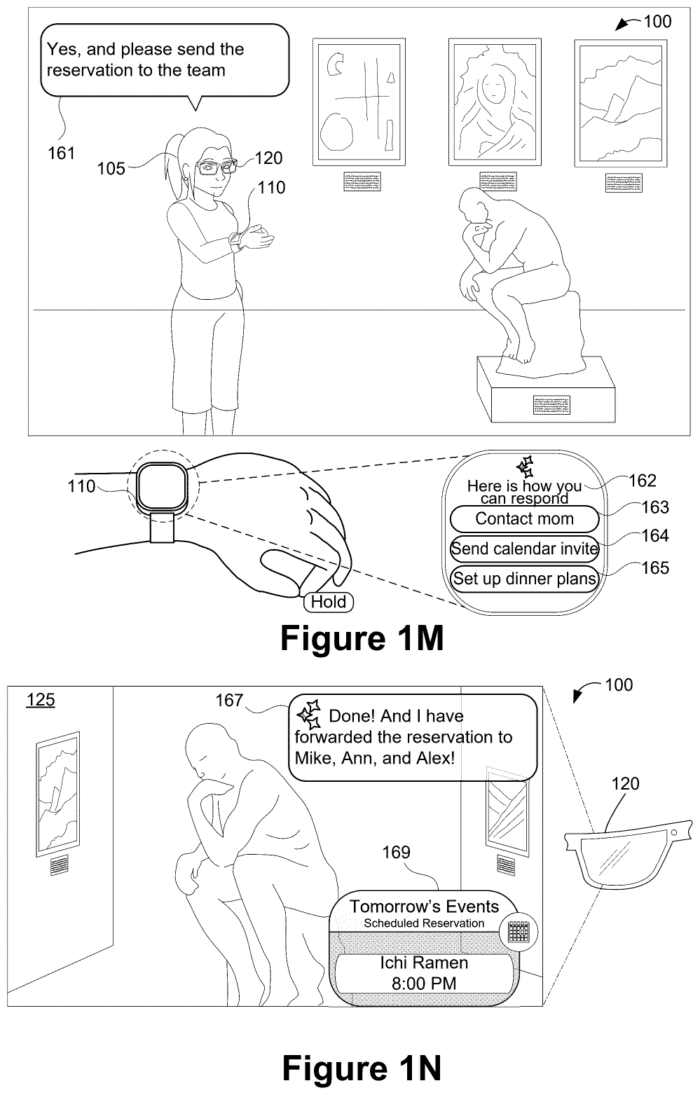

FIGS. 1A-IN illustrate invocation of an artificially intelligent agent at one or more wearable devices for providing guidance based on an activity of a user, in accordance with some embodiments. An AI guidance system 100 shown and described in reference to FIGS. 1A-IN provides example orchestrated guidance provided to a user 105 visiting a museum. The AI guidance system 100 includes at least a wrist-wearable device 110 and a head-wearable device 120 donned by the user 105. The AI guidance system 100 can include other wearable devices worn by the user 105, such as smart textile-based garments (e.g., wearable bands, shirts, etc.), and/or other electronic devices, such as an HIPD 742, a computer 740 (e.g., a laptop), mobile devices 750 (e.g., smartphones, tablets), and/or other electronic devices described below in reference to FIGS. 7A-7C. The AI guidance system 100, the wearable devices, and the electronic devices can be communicatively coupled via a network (e.g., cellular, near field, Wi-Fi, personal area network, wireless LAN). The AI guidance system 100 further includes an AI agent 115 (represented by star symbols) that can be invoked by the user 105 via one or more devices of the AI guidance system 100 (e.g., a wearable device, such as a wrist-wearable device 110 and/or a head-wearable device 120). Alternatively or in addition, in some embodiments, the AI agent 115 can be invoked in accordance with a determination that an AI agent trigger condition is present (as discussed below).

As described below in reference to FIG. 7A, the wrist-wearable device 110 (analogous to wrist-wearable device 726; FIGS. 7A-7C-2) can include a display 112, an imaging device 114 (e.g., a camera), a microphone, a speaker, input surfaces (e.g., touch input surfaces, mechanical inputs, etc.), and one or more sensors (e.g., biopotential sensors (e.g., EMG sensors), proximity sensors, ToF sensors, sensors of an IMU, capacitive sensors, strain sensors, etc.). Similarly, the head-wearable device 120 (analogous to AR device 728 and MR device 732; FIGS. 7A-7C-2) can include another imaging device 122, an additional microphone, an additional speaker, additional input surfaces (e.g., touch input surfaces, mechanical inputs, etc.), and one or more additional sensors (e.g., biopotential sensors (e.g., EMG sensors), gaze trackers, proximity sensors, ToF sensors, sensors of an IMU, capacitive sensors, strain sensors, etc.). In some embodiments, the head-wearable device 120 includes a display.

Turning to FIG. 1A, the wrist-wearable device 110 provides first example orchestrated guidance. While the user 105 is at the museum, the wrist-wearable device 110 and the head-wearable device 120 capture at least sensor data and image data via one or more sensors and/or imaging devices (e.g., imaging devices 114 and 122). In some embodiments, the head-wearable device 120 captures audio data. The AI guidance system 100 can determine, based on image data, sensor data, audio data, and/or any other data available to the AI guidance system 100, whether an AI agent trigger condition is satisfied and, in accordance with a determination that an AI agent trigger condition is satisfied, the AI guidance system 100 can provide the indication that an AI agent trigger condition is present. In response to an indication that an AI agent trigger condition is present, the AI guidance system 100 provides the AI agent 115, at least, image data, sensor data, audio data, and/or any other data captured by the devices of the AI guidance system 100. Alternatively or in addition, in some embodiments, the AI guidance system 100 provides the AI agent 115, at least, image data, sensor data, audio data, and/or any other data captured by the devices of the AI guidance system 100 in response to user invocation of the AI agent 115. The AI agent 115 can be invoked via touch inputs, voice commands, hand gestures detected by and/or received at the wrist-wearable device 110, the head-wearable device 120, and/or any other device of the AI guidance system 100.

The AI agent 115 can use, at least, the image data and/or the sensor data received from the AI guidance system 100 to determine a context-based activity. For example, the AI agent 115 can use the image data and/or the sensor data to determine that the user 115 is visiting or exploring the museum. In some embodiments, the AI agent 115 can also use audio data to determine a context-based activity. The context-based activity can be a physical activity (e.g. running, walking, etc.) and/or participation in an event (e.g., sightseeing, performing a hobby, cooking, driving, participating in a meeting, etc.). The AI agent 115 can further generate orchestrated guidance based on the context-based activity. The orchestrated guidance includes a recommended action for performing the context-based activity. The AI guidance system 100 can present the orchestrated guidance at a wearable device (e.g., the wrist-wearable device 110 and/or the head-wearable device 120) and/or any other communicatively coupled device.

For example, in FIG. 1A, the AI agent 115 provides orchestrated guidance for the user 105's museum visit, the orchestrated guidance including one or more recommended actions for facilitating the museum visit. The orchestrated guidance and the recommended actions are presented at a display 112 of the wrist-wearable device 110. In FIG. 1A, the wrist-wearable device 110 presents, via the display 112, the first orchestrated guidance 116 (e.g., “Welcome to the museum! Here are some things you can do!”) and the recommended actions (e.g. take tour user interface (UI) element 118 and do-not-disturb UI element 119) generated by the AI agent 115. In this way, the AI guidance system 100 can tailor the guided tour for the user 105.

FIG. 1B shows a field of view 125 of the user 105 via the head-wearable device 120. As shown in FIG. 1B, the orchestrated guidance generated by the AI agent 115 can also be presented via a display of the head-wearable device 120. For example, the field of view 125 of the user 105 includes a first orchestrated guidance UI element 127 (e.g. “Welcome to the museum! Let's take a look around”). While FIGS. 1A and 1B show orchestrated guidance and recommended actions presented at displays of the wrist-wearable device 110 and/or the head-wearable device 120, in some embodiments, the orchestrated guidance and recommended actions can be presented via a speaker of wrist-wearable device 110, the head-wearable device 120, and/or another communicatively coupled device.

FIG. 1C show the user 105 providing a first user input 129 selecting a recommended action of the first orchestrated guidance 116. In particular, the user 105 performs a hand gesture (e.g. a pinch) to provide a first user input 129 selecting the do-not-disturb UI element 119. In some embodiments, the first user input 129 selecting the do-not-disturb UI element 119 causes the wrist-wearable device 110, the head-wearable device 120, and/or other devices of the AI guidance system 100 to initiate a do-not-disturb mode (or focus mode, away mode, etc.). While in the do-not-disturb mode, the AI guidance system 100 suppresses, at least, received notifications, calls, and/or messages. In some embodiments, the use 105 can provide a voice request and/or other input to the AI guidance system 100 to silence notifications and provide a summary of the notifications later.

FIG. 1D shows a confirmation message generated by the AI agent 115. The AI agent, in response to the first user input 129, generates a corresponding response or recommended action. For example, the field of view 125 of the user 105 includes a confirmation message UI element 130 based on an accepted recommended action of the first orchestrated guidance 116.

FIG. 1E shows updates to the first orchestrated guidance 116 based on one or more user inputs. The orchestrated guidance generated by the AI agent 115 can include a subset of a plurality of recommended actions for performing the context-based activity. The orchestrated guidance, when presented at a wearable device, can include at least the subset of the plurality of recommended actions for performing the context-based activity. In some embodiments, one or more recommended actions of an orchestrated guidance are updated based on a user input selecting the one or more recommended actions. For example, the first orchestrated guidance 116 includes at least two UI elements—take tour UI element 118 and do-not-disturb UI element 119—and the AI agent 115 updates the first orchestrated guidance 116 to replace the do-not-disturb UI element 119 with a view map UI element 131 after detecting the first user input 129 selecting the do-not-disturb UI element 119. Similarly, the second user input 133 selecting the take tour UI element 118 cause the AI agent 115 to present updated first orchestrated guidance 116 and/or updated recommended actions. Alternatively, or in addition, in some embodiments, one or more recommended actions of an orchestrated guidance are updated based on the user 105 forgoing to select or ignoring one or more recommended actions.

In some embodiments, the AI agent 115 can determine that a context-based activity is one of a plurality of context based activities and, when generating the orchestrated guidance, determine a sequence for performing the plurality of context based activities (or context-based activities to be performed together and/or on parallel). For example, the context-based activity can be a first context-based activity of a plurality of context-based activities determined by the by the AI agent 115 (based on the sensor data, audio data, and/or image data), the orchestrated guidance can include a plurality of recommended actions for performing the plurality of context-based activities, and the recommended action is a first recommended action of the plurality of recommended actions, the first recommended action being configured to perform the first context-based activity.

In some embodiments, the AI agent 115 can determine when one or more context-based activities are completed, identify similar context-based activities, provide alternate context-based activities (if one or more specific context-based activities cannot be performed or alternate suggestion are present). For example, the user 105 can have a schedule including at least two events—the museum visit (e.g., a first context-based activity) and a dinner (e.g., a second context-based activity)—and the orchestrated guidance determined by the AI agent 115 can include a first set of recommended actions for augmenting the user 105's museum visit and a second set of recommended actions for augmenting the user 105's dinner, the second set of recommended actions being presented to the user 105 in accordance with a determination that the museum visit has concluded (e.g., the user 105 leaves the museum, the user 105 terminates an augmented experience for the museum visit provided by the AI agent, the scheduled museum visit time elapses, etc.).

FIG. 1F shows a context-based response generated by the AI agent 115. The context-based response is generated in response to the second user input 133 selecting the take tour UI element 118. In particular, the AI agent 115 generates a context-based response to facilitate the museum tour. For example, the user 105 can view a piece of art and the AI agent 115 can recognize the art and provide contextual information (or the context-based response) to the user 105 (e.g., by presenting the information at a wrist-wearable device). The AI agent 115 can use the sensor data, audio data, and/or the image data to generate the context-based response. For example, in FIG. 1F, the AI agent 115 uses the sensor data, audio data, and/or the image data to identify that the statute 134 is an object of interest to the user 105 and generates a context-based response based on the statute 134. Identification of an object of interest is discussed below in reference to FIG. 1G.

The context-based response can be presented at the wrist-wearable device 110, the head-wearable device 120, and/or any device communicatively coupled to the AI guidance system 100. For example, the AI agent 115 presents a first context-based response UI element 135 via a display of the head-wearable device 120, as shown in field of view 125 of the user 105.

FIG. 1G shows identification of an object of interest. In some embodiments, the AI guidance system 100 can identify an object of interest based on user gaze (determined by one or more eye tackers, sensors, and/or imaging devices of the head-wearable device 120 (e.g., gaze of user focused on an object for a predetermined amount of time (e.g., 10 seconds, 30 seconds, etc.))), direction of a field of view of the user 105 (determined by one or more sensors and/or imaging devices of the head-wearable device 120), pointing gestures performed by the user 105 (determined by one or more sensors and/or imaging devices of the wrist-wearable device 110 and/or the head-wearable device 120), voice commands, and/or other inputs provided by the user 105 to select an object of interest. For example, in FIGS. 1G, the user 105 provides a voice command 137 describing an object of interest. Alternatively, or in addition, the user 105 can perform a pointing gesture 138 to identify the object of interest and/or to augment or supplement the voice command 137. In other words, the AI guidance system 100 can use one or more inputs modalities to identify an object of interest. In this way, the AI guidance system 100 can provide the user 105 with a tailored guided tour of a venue or the museum based on user specific objects of interest (animate or inanimate) within the venue or the museum (e.g., artwork the user 105 spends time appreciating).

FIG. 1H shows one or more additional UI elements associated with the orchestrated guidance. In some embodiments, the AI guidance system 100 can present a highlight and/or one or more animations to identify a target object or object of interest. The head-wearable device 120 can include a dimmable lens controlled by the AI guidance system 100 and can provide additional information to the user 105 (e.g., directing the user 105's focus to certain objects within their field of view). For example, in FIG. 1H, the AI guidance system 100 cause selective dimming of a portion of a display of the head-wearable device 120 such that an animated dimming target UI element 139 is presented to the user 105. The animated dimming target UI element 139 can be used to draw the user 105's attention to a portion of the field of view 125 such that the use 105 can confirm a selected object of interest or be notified of a portion of the field of view 125 being analyzed by the AI guidance system 100.

FIG. 1H further shows a second context-based response presented at the wrist-wearable device 110, the head-wearable device 120, and/or any device communicatively coupled to the AI guidance system 100. For example, the AI agent 115 presents a second context-based response UI element 141 via a display of the head-wearable device 120, as shown in field of view 125 of the user 105. The second context-based response is based on the object of interest identified by the user 105 and highlighted by the AI guidance system 100. The context-based responses can also be provided as audio responses (or audio guidance) via speakers of the wrist-wearable device 110, the head-wearable device 120, and/or any device communicatively coupled to the AI guidance system 100.

Turning to FIG. 1I, updated orchestrated guidance is presented to the user 105. In particular, second orchestrated guidance 143 including a second set of recommended actions e.g., UI elements 144, 145, and 146) are presented to the user 105 via one or more wearable devices. The second orchestrated guidance 143 and the second set of recommended actions can be based on the user's current and/or past experiences at the museum and/or during the museum tour. For example, in accordance with a determination by the AI guidance system 100 that the user 105 has not previously viewed landmarks near the museum, the AI agent 115 can provide a recommended action to explore the unseen landmarks (e.g., as shown by explore landmarks UI element 145). As further shown in FIG. 1I, the user 105 provides a third user input 147 selecting an end tour UI element 146.

FIG. 1J shows a notification summary presented at a wearable device of the AI guidance system 100. In some embodiments, the AI guidance system 100, in accordance with a determination that the end tour UI element 146 was selected, ceases the user 105's participation in the context-based activity (e.g., the museum visit). The AI guidance system 100, in accordance with a determination that the museum visit has ended, causes the wearable devices or other communicatively coupled devices to cease the do-not-disturb mode. The AI guidance system 100, after detecting that the do-not-disturb mode ceased, generate, using the AI agent 115, a notification summary based on the notifications received while the wearable devices (or other devices of the AI guidance system 100) were in the do-not-disturb mode. In some embodiments, the summary can be a natural language summary provided by the AI agent 115 that summarized the received notifications. The notification summary can be presented via visual feedback (e.g., notification summary UI element 140 presented via a communicatively coupled display), audio feedback (e.g., text-to-speech presented via a communicatively coupled speaker), and/or haptic feedback.

FIG. 1K shows further updated orchestrated guidance. In particular, FIG. 1K shows a third orchestrated guidance 153 and a third set of recommended actions (e.g., UI elements 154 and 155) presented at a wearable device. The AI agent 115 determines the third orchestrated guidance 153 and the third set of recommended actions based on the notifications received while the wearable devices (or other devices of the AI guidance system 100) were in the do-not-disturb mode. For example, the third orchestrated guidance 153 and the third set of recommended actions provide the user 105 with options for responding to received messages and missed calls.

As further shown in FIG. 1K, the user 105 forgoes selecting the third set of recommended actions. Alternatively, the user 105 provides a touch input 157 at the head-wearable device 120 to initiate a microphone of the head-wearable device 120 (or other communicatively coupled device) and provide a voice command 151 to the AI guidance system 100. The voice command provided to the AI guidance system 100 can be used by the AI agent to determine another context-based activity (e.g., organizing dinner plans). The AI agent 115 can generate for the other context-based activity additional orchestrated guidance recommended action for performing the other context-based activity. For example, the AI agent 115 can generate orchestrated guidance for organizing dinner plans and recommended actions.

FIG. 1L shows the AI guidance system 100 utilizing a web-agent to assist the user in the performance of the other context-based activity and/or determine recommended actions. In some embodiments, in response to a user input selecting the recommended action for performing the context-based activity, the AI guidance system 100 can perform, using the AI agent, a (web or application) search based on the recommended action. The AI guidance system 100 can further determine a task to perform based on the search, and presenting the task at the wearable device. For example, in some embodiments, the AI guidance system 100 receives a request from a user to cause an AI agent to perform a task (e.g., “find a restaurant for dinner tomorrow downtown and make a reservation for 4”) and, based on content of the request, the AI guidance system 100 can determine that traversal of one or more web pages is required to perform the task that fulfills the request from the user. Further, the AI guidance system 100, responsive to the request, can traverse, using a web-based AI agent, one or more web pages and/or applications and, after the traversing, process the collected data to generate, via the AI agent, the response for the user 105 (e.g., response identifying a restaurant for 4 people and a time for making reservations).

In some embodiments, the AI guidance system 100 use the web-agent to autonomously carry out requests made by the user 105 even when the request is not associated with an API. In some embodiments, the AI guidance system 100 will report back on progress made in fulfilling the request of the user 105. For example, the AI guidance system 100 can report to the user 105 restaurant availability, restaurant wait times, errors in booking, reservation confirmations, etc. For example, as shown in FIG. 1L, the AI agent 115 identifies a restaurant and a reservation time for organizing the user 105's dinner plans, and the AI guidance system 100 presents the restaurant and the reservation time to the user 105 via the wearable device (e.g., response UI element 159).

In some embodiments, the AI guidance system 100 can utilize the web-agent (application-agent and/or other computer implemented agent) to assist the user 105 in collecting additional information for fulfilling the request from the user 105. For example, the AI guidance system 100 can search information related to social media posts to identify restaurant recommendations and/or restaurants in proximity and provide the information related to the social media posts to the AI agent 115 for generating a response and/or providing recommended actions. In some embodiments, the information is determined through the use of an AI model that is configured to determine additional information from images/videos/audio to provide contextual information (e.g., a picture of a posted restaurant and use an AI to determine which restaurant the poster was at). In some embodiments, the AI guidance system 100 can provide the user 105 with information about a previously seen social media post. In some embodiments, the AI guidance system 100 can be used to find additional information on posts or other content the user 105 has previously viewed via one or more devices, thereby providing unique results specific to the user's viewing history.

In some embodiments, the AI guidance system 100 can perform additional AI actions to assist the user 105 and/or augment the user 105's experience. For example, the AI guidance system 100 can proactively provide or silence notifications based on user situations determined by the AI agent 115 (e.g. the AI guidance system 100 can detect ongoing activities of the user 105 based on sensor data, audio data, and/or image data, and determine situations would benefit from additional focus (e.g., productivity tasks, participation in events, etc.) and silence non-essential notifications until the situations are complete). Additionally, the AI guidance system 100 can also proactively display information that is determined to be essential to the user 105 and/or predicted to be useful to the user 105 based on the environment of the wearable devices and/or other devices of the AI guidance system 100. For example, a wearable device, such as the head-wearable device 120, can automatically display a menu of a restaurant (that is determined to be of interest to the user 105) when the user 105 is in proximity (e.g., 3 feet, 6 feet, etc.) of the restaurant such that the user 105 does not have to perform an additional search (e.g. navigate a search engine to find the menu). In some embodiments, the AI guidance system 100 operations can occur without the need of user input (e.g., touch inputs, voice commands, etc.).

FIG. 1M illustrate orchestrated guidance based on the restaurant identified by the AI guidance system 100. In particular, the AI guidance system 100 presents via the wearable devices a fourth orchestrated guidance 162 and a fourth set of recommended actions (e.g., UI elements 163, 164, and 165). In FIG. 1M, the user 105 provides another voice command 161 to the AI guidance system 100 for performing an action corresponding to the orchestrated guidance for organizing dinner plans. The user 105 performs a pinch and hold gesture to initiate a microphone of the head-wearable device 120 (or other communicatively coupled device) and provide the other voice command 161 to the AI guidance system 100.

FIG. 1N shows the AI guidance system 100 providing confirmation of a completed task and generating an event for the user 105. For example, the AI guidance system 100 causes presentation of a task completion UI element 167 via a display of the head-wearable device 120. Additionally, the AI guidance system 100 also presents a calendar UI element 169 showing an event or calendar invite generated by the AI agent 115.

The examples provided above are non-limiting. The AI guidance system 100 can be used to augment user experience of other activities. For example, the AI guidance system 100 can be used for a cooking context-based activity and the AI guidance system 100 can be used by the user 105 to find a recipe, make a dish based on the recipe, present guidance on preparation of the dish based on the recipe (e.g., step-by-step instructions, illustration, and/or video). Similar to the process described above, the AI guidance system 100 can use sensor data, audio data, and/or image data of wearable devices and/or other devices to determine a current step of the recipe and/or progress made by the user 105. For example, the user 105 can query the AI guidance system 100 on the next step of the recipe, and the AI guidance system 100 can provide tailored instructions to the user 105. In some embodiments, the AI guidance system 100 can provide information about steps of the recipe, how much time is left, determinations of food preparedness based on sensor data, audio data, image data, etc.

In another example, the AI guidance system 100 can augment a user experience of a game application. For example, a user can query the AI guidance system 100 to perform a task in game, and the AI guidance system 100 can leverage the one or more sensors of the wearable devices (e.g., the head-wearable device 120) and/or other devices in communication with the AI guidance system 100 to satisfy the request of the user 105. For example, the AI guidance system 100 can provide natural language responses to guide a user 105 within an augmented reality environment by using IMU data and image data (e.g., the device can state “There is a monster behind you, watch out!”). In some embodiments, the request to the AI guidance system 100 can initiate the game without the need for the user 105 to open the application themselves. In some embodiments, the AI guidance system 100 could output audio spatially to the user to help them identify where an interactable object is in a game.

In yet another example, the AI guidance system 100 can augment a user experience of a sports event or sports application. For example, the user 105 can ask the AI guidance system 100 a question about an ongoing Formula 1 race to understand the positions of the drivers—e.g., “compare the pace between two drivers.” The AI guidance system 100 can be configured to use live data from the application or the sports stream to provide the appropriate response. For sports that are heavily data driven, there is a lot of data that is not provided to the user 105, but the AI guidance system 100 can access any available data (e.g., microphone communications of one driver, tire data, lap times, showing different cameras of different drivers including selecting specific cameras on each car, etc.).

### Artificially Intelligent Context-Based Responses for User Activities

FIGS. 2A-2R illustrate context-based responses generated by an artificially intelligent agent based on activities performed by a user, in accordance with some embodiments. Similar to FIGS. 1A-IN, the operations shown in FIG. 2A-2R can be performed by any XR systems described below in reference to FIGS. 7A-7C. For example, the operations of FIGS. 2A-2R can be performed by wearable devices, such as a wrist-wearable device 110 and/or a head-wearable device 120. The operations of FIGS. 2A-2R are performed by an AI assistive system 200 including at least a wrist-wearable device 110 and a head-wearable device 120 donned by the user 105 and/or other electronic devices described below in reference to FIGS. 7A-7C. The AI assistive system 200 can include the AI agent 115. The AI assistive system 200 is analogous to the AI guidance system 100 shown and described in reference to FIGS. 1A-IN. In some embodiments, the AI assistive system 200 and the AI guidance system 100 are the same. Alternatively, in some embodiments, the AI assistive system 200 and the AI guidance system 100 are distinct system implemented at any XR systems described below in reference to FIGS. 7A-7C. Operations of the AI assistive system 200 and the AI guidance system 100 can be performed in parallel, sequentially, concurrently, and/or in a predetermined order.

In some embodiments, the AI assistive system 200 can augment the user 105's experience in performing a physical activity and/or user experience while using a fitness application. The AI assistive system 200 can assist the use in the performance of different physical or fitness activities. The AI assistive system 200 can operate as a virtual coach and emulate a coach's voice, provide specific instructions, and/or provide feedback to the user 105. For example, one or more sensors of the head-wearable device 120 and/or communicative coupled devices to determine whether the user 105 is performing the physical activity correctly. In accordance with a determination that the user 105 is not performing the exercise correctly, the AI assistive system 200 can provide guidance to the user 105 to improve performance of the exercise.

In FIGS. 2A-2R, the user 105 is participating in an activity with at least one other user 205. In some embodiments, the activity is physical exercise. For example, the user 105 and the at least one other user 205 are at a gym and start performing an exercise (e.g. a run). The AI assistive system 200, in response to an indication that the user 105 of a wearable device, such as the wrist-wearable device 110 and/or the head-wearable device 120, is participating in an activity, obtains data associated with an on-going activity performed by the user 105 of the wearable device. In some embodiments, the indication can be provided in response to a user input. For example, first user input 207 at the head-wearable device 120 initiating a workout. Alternatively, the AI assistive system 200 can generate the indication based on sensor data, audio data, and/or image data captured by one or more devices of the AI assistive system 200. For example, the AI assistive system 200 can detect that the user 105 is engaging in a physical activity, such as running, cycling, weightlifting, skiing, etc., and generate the indication that the user 105 is participating in an activity. In some embodiments, the AI assistive system 200 can generate the indication based on audio cues or context. For example, the user comment “ready for the run?” can be used to initiate and identify the activity.

The AI assistive system 200 generates, using the AI agent 115, a context-based response based, in part, on the data associated with the on-going activity performed by the user 105 of the wearable device and presents, at the wearable device, the context-based response. For example, as shown in FIG. 2B, the AI agent 115 can generate a workout UI 211 including activity information and a first context-based response (represented by first context-based response UI element 209), and cause presentation of the workout UI 211 and the first context-based response UI element 209 at the head-wearable device 120. In some embodiments, the context-based response is presented within a portion of a field of view 212 of the user 105. In some embodiments, the context-based response and/or the workout UI 211 are presented such that they are always visible to the user 105. For example, the context-based response and/or the workout UI 211 can be positioned at a portion of the display of the wearable device reserved for the context-based response and/or the workout UI 211. Alternatively, or in addition, the context-based response and/or the workout UI 211 can be configured such that they are always overlayed over other applications and/or UIs.

In some embodiments, the context-based response is a coaching response to assist the user 105 on performance of the activity. For example, in FIG. 2B, the first context-based response UI element 209 prompts the user 105 if they would like help with their workout. In some embodiments, the context-based response can include navigation instructions.

In some embodiments, the workout UI 211 includes activity information, such as activity information UI element 212 and activity route 217 (or activity map). In some embodiments, the workout UI 211 includes biometric data to allow the user 105 to easily track their workout. For example, the workout UI 211 can include real-time statistics including, but not limited to, speed, pace, splits, total distance, total duration, map, segments, elevation, gradient, heart rate, cadence, persona records (or PRs), challenges, and segment comparisons. In some embodiments, the AI assistive system 200 operates in conjunction with the wearable devices to automatically select information about the physical activity to present within the user interface elements. In some embodiments, the workout UI 211 includes one or more quick access applications 215 that allow the user 105 to initiate one or more applications.

The AI assistive system 200 can present and/or share data rich overlay UIs that can include image data (e.g., FIGS. 2L and 2C) and/or other data about activities that the user 105 is performing. The AI assistive system 200 allows the user 105 to connect and engage with their communities in more interesting and engaging ways, by curating informative overlays to captured activities. For example, by providing the user 105 with capabilities for sharing personal states about physical activities that the user is performing, the AI assistive system 200 allows the user 105 to elevate and showcase their efforts and progress.

In some embodiments, the AI assistive system 200 can provide visual feedback to the user 105 via frames of the head-wearable device 120. For example, the head-wearable device 120 includes one or more indicators for assisting the user 105 in performance of the activity. For example, FIG. 2B shows an interior portion 219 (e.g., face-facing portion of the frames) of the head-wearable device 120, the interior portion including a first light emitter portion 221 and a second light emitter portion 223. The first and the second light emitter portions 221 and 223 can be light-emitting diodes (LEDs). The AI assistive system 200 can use the first light emitter portion 221 and the second light emitter portion 223 to provide directions to the user (e.g., turn the first light emitter portion 221 on and the second light emitter portion 223 off to direct the user to the left, turn on both the first and the second light emitter portions 221 and 223 to direct the user to go forward; etc.). In some embodiments, the first and the second light emitter portions 221 and 223 can turn different colors, illuminate in different patterns and/or frequencies, and/or illuminate with different brightness to provide the user 105 with biometric information (e.g., green to indicate that the heart rate of the user 105 is in a first target threshold, yellow to indicate that the heart rate of the user 105 is in a second target threshold, red to indicate that the heart rate of the user 105 is in a third target threshold, etc.)

In FIG. 2C, the user 105 responds to the first context-based response via a voice command 225. In particular, the user 105 requests that the AI assistive system 200 assist the user 105 in setting a PR. The user 105 can provide different types of request to the AI assistive system 200. For example, the user 105 can provide the voice command 225 requesting that the AI agent 115 notify the user 105 when their heart rate is above a predefined threshold (e.g., heart rate goes above 165 BPM). The AI assistive system 200 can provides a series of visual and/or audio response to the user 105 based on the voice command 225 or other user request. The visual and/or audio response can be encouragement, suggestions, instructions, updates to biometric data, etc. In some embodiments, the AI assistive system 200 can provide the audio response in distinct vocal personalities and/or other characteristics, which may be based on the type of physical activity the user is performing (e.g., a personified AI agent). For example, the AI assistive system 200 can use the voice of a famous motivational runner in accordance with detecting that the user 105 is running a 10K.

In FIG. 2D, the AI assistive system 200 generates, via the AI agent 115, second context-based response and updates to the workout UI 211. For example, the AI assistive system 200 can generate a response to the voice command 225 and present the response to the user 105 via a wearable device (e.g., the second context-based response UI element 227 presented within field of view 212). Additionally, the workout UI 211 can be updated to show changes to biometric data (e.g., changes to calories burned, heart rate, etc.), workout completion, split times, etc.

In some embodiments, the AI assistive system 200 generates, via the AI agent 115, proactive alerts. The proactive alerts are custom alerts that help a user 105 stay aware of their performance metrics and train toward specific goals. In some embodiments, the user 105 can customize their alerts on an electronic device (e.g., mobile device 750; FIGS. 7A-7C) and/or one or more applications running on a device of the AI assistive system 200 and/or an XR system. The one or more alerts can be provided via any device of the AI assistive system 200 and/or an XR system and/or program (or application) running on a device of the AI assistive system 200 and/or an XR system. The proactive alerts notify the user 105 about specific events or data, such as whether the user 105 is below, above, or at a set threshold.

For example, in some embodiments, the AI assistive system 200, in accordance with a determination that one or more progress triggers are satisfied, generates, by the AI agent 115, physical activity notification (e.g., a proactive alert) including one or more of real-time metrics, personal goal progress, and physical activity progress. The AI assistive system 200 further causes presentation of the physical activity notification via the head-wearable device 120, the wrist-wearable device 110, and/or any other communicatively coupled device. The one or more progress triggers can include one or more of an event trigger, a predetermined threshold, and a predetermined interval. In some embodiments, the event trigger is a specific trigger defined by the user 105, such as a landmark, reaches a predefined pace, reaches a predefined workout elapsed time, rest for a predefined time, etc. The predetermined threshold can be any predefined value and/or predefined range, such as heart rate between in predefined minimum and predefined maximum values, speed above a predefined threshold, etc. The predetermined interval can be any recurring values, such as every 5 minutes, every 1 mile, any lap around a course or running trail (e.g., detected via image date).

Turning to FIG. 2E, the user 105 provides the AI assistive system 200 a request to live stream their activity. The AI assistive system 200 can allow the user 105 to enable a live stream using wearable devices (e.g., the head-wearable device 120 and/or the wrist-wearable device 110) and/or other communicatively coupled device capture and transmit image data, audio data, and/or sensor data. For example, as shown in FIG. 2E, the user 105 can provide another voice command 229 requesting that the AI assistive system 200 initiate a stream to capture their run. In some embodiments, the user 105 can initiate the live stream via a touch input at the wrist-wearable device 110 and/or the head-wearable device 120. In some embodiments, the user 105 can perform a gesture to select one or more UI element for selecting a particular functionality. For example, the user can perform a pinch gesture to select the streaming UI element 234.

In FIG. 2F, the AI assistive system 200 provides a third context-based response confirming the initiation of the stream to the user 105 (e.g., third context-based response UI element 231). The AI assistive system 200 can further present a streaming UI 233 at the head-wearable device 120 and/or another streaming UI 237 at the wrist-wearable device (or other communicatively coupled display). In some embodiments, the AI assistive system 200 can present static holographic elements 235 that provide simple information and/or images to the user 105. For example, the static holographic elements 235 can includes battery information, simplified notifications corresponding to stream interactions, and/or other AI agent 115 information (such as a camera view finder) can be presented.

The AI assistive system 200 can initiate the live stream on one or more platforms associated with the user 105. In some embodiments, the AI assistive system 200 can automatically select the streaming platform for the user 105 (e.g., based on user behavior). Alternatively, or in addition, the user 105 can provide a user input (e.g., voice command, touch input, gesture, etc.) identifying a streaming platform and/or selecting from one or more suggested streaming platforms identified by the AI assistive system 200. In some embodiments, the AI assistive system 200 notifies one or more followers of the user 105 that the live stream has been initiated. In other words, in some embodiments, the AI assistive system 200 can perform a complimentary operations to a requested operation of the user 105, which may be based on data about the user's interaction history with the respective social platforms.

In some embodiments, the streaming UI 233 and the other streaming UI 237 include a chat of the live stream. Alternative, or in addition, the streaming UI 233 and the other streaming UI 237 can present the broadcasted stream (e.g., captured and transmitted image data, audio data, sensor data, and/or other transmitted data). In some embodiments, the user 105 can toggle information presented via the streaming UI 233 and/or the other streaming UI 237. For example, the user 105 can select one or more UI elements within the streaming UI 233 and/or the other streaming UI 237 to toggle the presented information. Additionally, the user 105 can select a share UI element to share additional content or information. In some embodiments, the AI assistive system 200 can apply one or more overlays and/or UI elements to the streamed data such that the one or more overlays and/or UI elements are viewable by devices receiving the streamed data. For example, the streamed image data can include information on the user's current activity (e.g., current progress, percentage complete, and/or other information shared by the user 105). The AI assistive system 200 can provides automatic user interactions by automatically engaging the user 105 and/or with other communicatively coupled devices with streamed data.

FIGS. 2G and 2H shows the AI assistive system 200 connecting the user 105 with the at least one other user 205. In some embodiments, the AI assistive system 200, in accordance with a determination that the activity is a group activity performed with at least one contact of the user 105 (e.g., a friend or connection of the user 105), obtains from an electronic device associated with the at least one contact of the user 105 additional data associated with a respective on-going activity performed by the at least one contact of the user 105. The context-based response can further be based on the additional data associated with the respective on-going activity performed by the at least one contact of the user. For example, as shown in FIG. 2G, the AI assistive system 200 presents via a display of the head-wearable device 120 a context-based response (e.g., a fourth context-based response 239) prompting the user 105 if they would like to connect with a contact (e.g., the at least one contact 205), as well as an updated workout UI 211 including a pin 241 or flag of a position of the at least one contact 205 relative to a current position of the user 105. FIG. 2H further shows the user 105 providing a user input (e.g., yet another voice command 243) requesting that data be shared with the at least one contact 205.

In some embodiments, the AI assistive system 200 provides a plurality communication modalities in which the user 105 can quickly connect with friends and/or contacts. The AI assistive system 200 can be used to contact a single contact participating in a group activity or all contacts participating in the group activity. In some embodiments, the AI assistive system 200 can include one or more communication channels. For example, the AI assistive system 200 can include a walkie-talkie feature to quickly and effortlessly connect with one or more contacts. In some embodiments, the AI assistive system 200 can identify one or more participants in a group activity based on proximity data of one or more devices adjacent to wearable devices of the AI assistive system 200. Alternatively, or in addition, in some embodiments, the AI assistive system 200 can identify one or more participants in a group activity based on electronic devices attempting to communicatively couple with the wearable devices and/or other devices of the AI assistive system 200. In some embodiments, the AI assistive system 200 can identify one or more participants in a group activity based on the user 105's contact list and/or by reviewing recent group conversations about an event or activity. In some embodiments, the AI assistive system 200 uses natural language systems to invoke a conversation with a group and quickly communicate with the group. For example, the user may invoke a conversation generally without specifying the recipients and based on what the user 105 asks, the AI assistive system 200 can determine the appropriate audience (e.g., asking “where is everyone?” when the user is at a food festival with friends).

FIGS. 2I and 2J show a perspective of the at least one contact 205. In particular, FIGS. 2I and 2J show another AI assistive system (analogous to the AI assistive system 200) implemented on one or more wearable devices or other devices of the at least one contact 205. In FIG. 2I, the other AI assistive system presents via a speaker of a head-wearable device 253 of the at least one contact 205 a context-based response 245 prompting the at least one contact 205 if they would like to connect with the user 105. The at least one contact 205 further provides a voice command confirming that they would like to connect with the user 105.

FIG. 2J shows a field of view 246 of the at least one contact 205 as viewed by the head-wearable device 253. The field of view 246 of the at least one contact 205 includes a first workout UI 249 tracking the at least one contact 205's workout and a second workout UI 247 including shared workout information from the user 105. The first workout UI 249 further includes a respective pin 250 identifying the location of the user 105 relative to the at least one contact 205. FIG. 2J further shows the at least one contact 205 providing the other AI assistive system a request. For example, the request 251 from the at least one contact 205 asks the other AI assistive system to send an encouraging message to user 105. In some embodiments, the AI assistive system 200 of the user 105 can receive the encouraging message and automatically cause presentation of the visual and/or audio message. In some embodiments, the encouraging message can include a haptic feedback response. In some embodiments, the AI assistive system 200 presents the encouraging message after determining that the user 105 has achieved a particular milestone related to the performance of the activity. In some embodiments, users are able to unlock pre-recorded praise from the AI assistive system 200 (e.g., personified AI agents) and/or pre-recorded audio by professional athletes related to the physical activities that the user is performing.

FIG. 2J further shows one or more indicators 255 on the head-wearable device 253 of the at least one contact 205. The indicators 255 of the head-wearable device 253 of the at least one contact 205 can be one or more light-emitters (e.g., LEDs). Similar to the first and second light emitter portions 221 and 223, the indicators 255 can communicate information to the at least one contact 205. For example, the indicators 255 can illuminate in different colors, patterns and/or frequencies, and/or brightness to convey information to the at least one contact 205. For example, the indicators 255 can illuminate to notify the at least one contact 205 when they are within target activity thresholds, performing an activity at a predetermined pace or speed, etc. In some embodiments, the indicators 255 provides a persistent indication to the at least one contact 205 based on whether a particular condition satisfies a predefined threshold. For example, based on the at least one contact 205 providing a user input activating the indicators 255, the indicators 255 can remain active until disabled.

In some embodiments, the head-wearable device 253 are a pair of low-cost head-wearable device that do not include a display and opt for presenting information via audio outputs and/or haptic feedback to the at least one contact 205. Alternatively, in some embodiments, the head-wearable device 253 can include low fidelity display that is configured to provide glanceable information. In some embodiments, this information may be text and glyphs (e.g., emoji's, gifs, or low-resolution images) only, as opposed to media rich images (e.g., video or color images). In some embodiments, the low-fidelity display can be configured to display a single color (e.g., green) or grayscale. In some embodiments, the head-wearable device 253 can include an outward facing projector configured for displaying information. For example, the head wearable device 253 can be configured to display a text message onto a wearer's hand or other surface. In some embodiments, the head-wearable device can project user interfaces such that a wearer can interact with a desktop-like user interface without needing to bring a laptop with them.

While these head-wearable devices are shown as having different features it is envisioned that a single head-wearable device could be configured to use all or a subset of these information presenting modalities, in accordance with some embodiments.

As described above, the AI assistive system 200 can include different modalities for presenting and/or sharing information. While numerous modalities are discussed, it is envisioned that an operating system would be configured to present the information based on the device, and the developer would only need to specify the content to be presented and not the specific modality. In this way software can be produced to work across head-wearable devices with different capabilities (e.g., information output modalities). All of these devices described are configured to work with AI models for presenting information to users.

FIGS. 2K and 2L show additional data collected and/or shared during the performance of an activity (or group activity). For example, FIGS. 2K and 2L show image data collected during the performance of the group activity, shared image data between the members of the group activity, and/or synchronization of the image data. In FIG. 2K, the other AI assistive system presents via a speaker or a display of the head-wearable device 253 of the at least one contact 205 another context-based response 257 prompting the at least one contact 205 if they would like receive and synchronize image data shared by the user 105. The at least one contact 205 further provides a voice command confirming that they would like to connect receive and synchronize image data shared by the user 105.

In other words, the AI assistive system 200 include sharing operations for creating and sharing user interfaces that include imaging data captured by the intelligent auto-capture assistive operations. The AI assistive system 200 provides user interfaces that include image data that is captured while a user performing a physical activity (e.g., a fitness activity, such as performing a bike ride). In addition to the image data, the user interfaces also include user interface elements generated based on other data, different than the image data, related to the user's performance of the respective physical activity. In some embodiments, the AI assistive system 200 is configured to allow users to tag captured media with personal metadata (e.g., real-time statistics). For example, the user interfaces may include engaging montages of captured images and other content about the performance of the physical activity. As shown in FIG. 2L, an image sync UI 259 can be configured display captured image data, combined image data (e.g., combined first image data 261 and second image data 263), and/or image montages. In some embodiments, the image sync UI 259 can be presented at other devices of the AI assistive system 200. In some embodiments, the AI assistive system 200, in accordance with a determination that a plurality of video streams are (i) captured within a predefined amount of time of each other and (ii) within a predefined distance of each other, prepares a collated video of two or more of the plurality of video streams in a time-synchronized fashion.

FIGS. 2M and 2N show the at least one contact 205 providing an additional request to the AI agent 115. In FIG. 2M, the at least one contact 205 request for insight and/or a summary regarding their current workout. In particular, the at least one contact 205 provides the AI agent 115 with an in-activity insight request 259 (e.g., “Assistant, how am I doing?”). The AI agent 115, in response to the in-activity insight request generates an AI message that summarizes personal activity data and provides insights. The insights can be provided in a positive tone to help a user progress toward their goals. The AI message includes real-time metric data, a physical activity summary, physical activity progress, user target progress, and historical performance information.

FIG. 2N shows an example AI message in response to the in-activity insight request. The AI message includes real-time metric data, a physical activity summary, physical activity progress, user target progress, and historical performance information. For example, an insight AI message 261 includes a summary of the at least one contact 205's run (e.g., current run distance, elapsed time, running pace, and target towards personal record. The at least one contact 205 can include real-time biometric data (e.g., hear rate, oxygen saturation, hydration levels, temperature, etc.), real-time activity data (e.g., workout duration, workout interval (e.g., rest, high impact, cooldown, etc.), progress data, comparison against a target and/or goals, and/or other data captured by one or more sensors and/or stored in a user workout profile. In some embodiments, the one or more goals and/or targets are provided by fitness applications and/or other programs running on devices of the AI assistive system 200.

FIGS. 2O and 2P show the at least one contact 205 providing another additional request to the AI agent 115. In FIG. 2O, the at least one contact 205 requesting for real-time and/or historical performance data. For example, the at least one contact 205 provides the AI agent 115 with a progress update 263 (e.g., “Assistant, how much have I run?”). This allows a user to get real-time and historical performance metrics while keeping their hands and eyes free and focused on their activity. As described above, the data can be captured by one or more devices of the AI assistive system 200 and/or applications and/or other programs running on devices of the AI assistive system 200.

FIG. 2P shows another example AI message in response to the progress update request. For example, a progress update AI message 265 includes a real-time update on the at least one contact 205's run distance. In some embodiments, the progress update AI message includes a real-time metrics on one or more of a distance, an elapsed time, a heart rate, an elevation, a location, power zones, a pace, a speed, split information, a cadence, and fitness application information.

FIGS. 2Q and 2R show a post-activity summary 271 generated by the AI agent 115. In some embodiments, the AI agent 115 generates the post-activity summary 271 automatically after the user 105 completes the activity. For example, the AI assistive system 200 in response to an indication that the user has ceased performing the physical activity, generates, by the AI agent, a physical activity summary (e.g., the post-activity summary 271). Alternative, in some embodiments, the AI agent 115 generates the post-activity summary 271 or updates the post-activity summary 271 in response to a user request. For example, as shown in FIG. 2Q, the user 105 provides the AI agent 115 with request to summarize their workout (e.g., summary request 267)

FIG. 2R shows an example of the post-activity summary 271. The post-activity summary 271 includes a summary of the user 105's workout including sensor data captured by during the physical activity. Additionally, in some embodiments, the post-activity summary 271 includes image data captured during the user's physical activity. As described herein, the image data can be captured by one or more devices of the AI assistive system 200, such as the head-wearable device 120, the wrist-wearable device 110, a mobile device 269 (e.g., analogous to mobile device 750; FIGS. 7A-7C), etc. In some embodiments, the image data is automatically captured when capture criteria is satisfied, as described herein. In some embodiments, the post-activity summary 271 is provided to a device of the AI assistive system 200 for display. For example, the post-activity summary 271 is presented via the mobile device 269.

The AI agent 115 takes large and complex sets of sensor data (e.g., physical activity data, biometric data, and/or other sensed data) and turns them into insightful the post-activity summary 271 that are easy to read and understand. The post-activity summary 271 are easy to share with user contacts, social media platforms, and/or with other electronic devices. In some embodiments, the post-activity summary 271 includes in-activity capture compilation with data overlays (e.g., image data and sensor data captured during the physical activity). The post-activity summary 271 allows for quick sharing to social media platforms and/or other applications and/or programs running on a device of the AI assistive system 200. In some embodiments, the post-activity summary 271 can be uploaded to fitness applications or other applications of the user. In some embodiments, the post-activity summary 271 is a high level summary generated based on activity data from (fitness) partner applications and/or devices.

### Example Context-Based Responses Provided by an Artificially Intelligent Agent

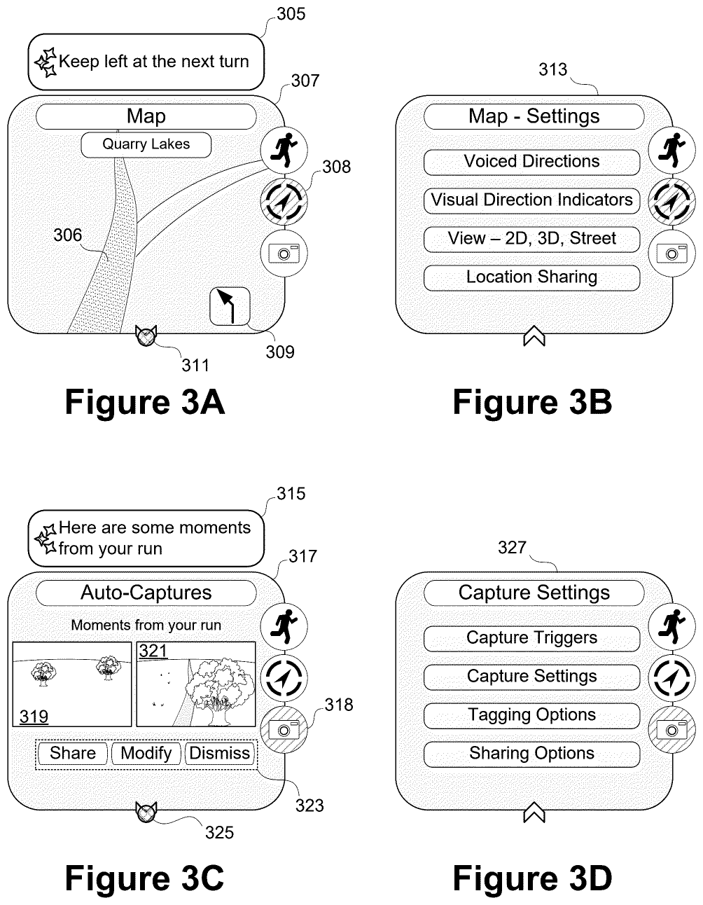

FIGS. 3A-3D illustrate example user interfaces and additional features available at the AI assistive system 200, in accordance with some embodiments. FIGS. 3A and 3B show a map application and directions provided via the AI assistive system 200. FIGS. 3C and 3D show automatic image capture capabilities of the AI assistive system 200.

In FIGS. 3A and 3B, the AI assistive system 200 presents a map UI 307. The map UI 307 can include one or more UI elements providing directions to the user 105. For example, the map UI 307 can include a next step UI element 309 including the next directions to take, as well as a path highlight 305 (which can be overlaid over the next path in the directions). In some embodiments, the user 105 can toggle between application via one or more user inputs. For example, the user 105 can cause presentation of the map UI 307, via a wearable device of the AI assistive system 200, in response to user selection of the map application UI element 308. Additionally, or alternatively, in some embodiments, the AI assistive system 200 presents context-based responses 305 providing directions to the user 105.

FIG. 3B shows a map settings UI 313. The map settings UI 313 can be presented in response to user input 311 (selecting the downward arrow). The map settings UI 313 provides one or more options for allowing the user 105 to select settings for voiced directions (e.g., on, off, and/or a particular voice), visual direction indicators (e.g., path highlights, next step UI elements, etc.), view (e.g., setting 2D, 3D, and/or street views), location sharing (e.g., privacy setting for sharing location, automatic sharing of location, etc.), etc.

Turning to FIGS. 3C and 3D, the AI assistive system 200 presents an image capture UI 317. The image capture UI 317 can include one or more UI elements for showing captured image data and/or options 323 for modifying, sharing, and/or dismissing the captured image data. For example, the image capture UI 317 can include first and second image data 319 and 321 captured during the activity of the user 105. In some embodiments, the user 105 can toggle between application via one or more user inputs. For example, the user 105 can cause presentation of the image capture UI 317, via a wearable device of the AI assistive system 200, in response to user selection of the image application UI element 318. Additionally, or alternatively, in some embodiments, the AI assistive system 200 presents context-based responses 315 providing information on the automatically captured image data.

FIG. 3D shows a capture settings UI 327. The capture settings UI 327 can be presented in response to user input 325 (selecting the downward arrow). The capture settings UI 327 provides one or more options for allowing the user 105 to select settings for capture triggers (e.g., triggers that cause the automatic capture of image data, such as changes in movement, instant spikes in acceleration, activity milestones (e.g., hitting a baseball with the baseball bat), changes in vibration, etc.), capture settings (e.g., image capture setting such as resolution, format, frames per second, etc.), tagging options (e.g., settings identifying people and/or objects to be tagged), sharing options (e.g., privacy setting for sharing image data, identifying images that can be shared, frequency at which image data is shared, etc.), etc. In some embodiments, the AI assistive system 200 is configured to perform sharing operations based on the user input in accordance with determining that the user has already enabled the automatic image-capture operations. In some embodiments, the AI assistive system 200 can perform automatic smoothing functions on image data.

### Example Interactions Using a Wearable Device Including an Artificially Intelligent Agent

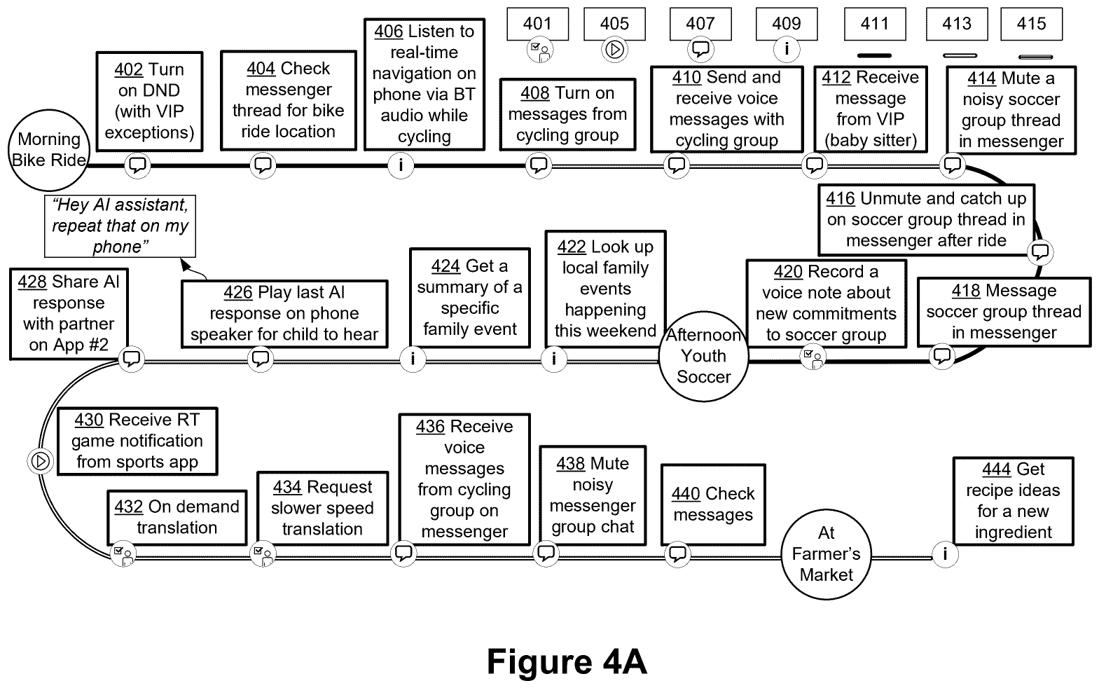

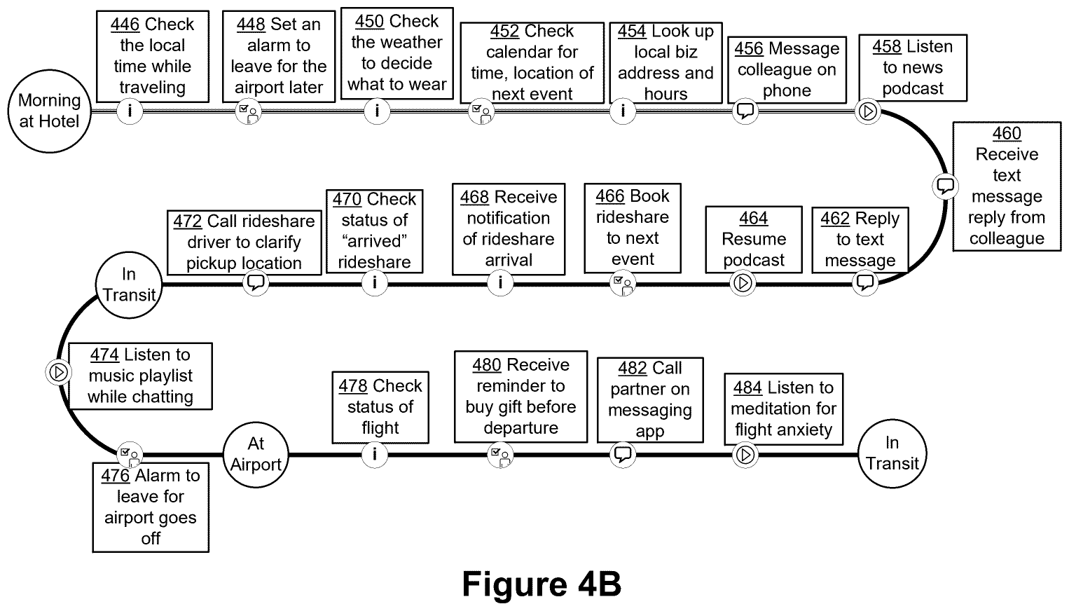

FIGS. 4A and 4B illustrate example sequences of user interactions with personalized assistive systems (e.g., the AI guidance system 100 and/or the AI assistive system 200; FIGS. 1A-2R), in accordance with some embodiments. The legend in the top right of FIGS. 4A and 4B indicates types of interactions and input modes for each respective segment of the timeline flow. The task icon 401 indicates a productivity-based interaction, media-play icon 405 indicates media and/or an “edutainment” interaction, the messaging icon 407 indicates a communication-based interaction, the information icon 409 indicates an information-based interaction, the solid line 411 indicates a touch input, the double line 413 indicates a wake word input, the triple line 415 indicates an AI chat session.

The interaction sequences of FIGS. 4A and 4B can be performed by a user that is wearing a head-worn device 120 (e.g., AR device 728) while the user of the device is performing a sequence of daily activities. The head-worn device 120 (FIGS. 1A-3D) includes or is in electronic communication with an assistive system for assisting in interactions with the head-worn device 120 to cause operations to be performed. For example, the head-worn device 120 may provide information (e.g., information related to data collected about a physical activity that a user is performing, an alert about an incoming message) without explicit user input to do so.

In accordance with some embodiments, the user can perform voice commands to cause operations to be performed at the head-worn device 120. For example, as shown in block 402, the user can provide a voice command to turn on do-not-disturb (DND) at their head-worn device 120, with an option set for VIP exceptions, which would allow for certain users' messages or other requests may be allowed. In some embodiments, the assistive system, in accordance with receiving the request to turn on do not disturb, determines a set of potential operation commands that the request may correspond to.

As shown in block 404, the assistive system can determine to check one or more messenger threads accessible via the head-worn device 120 to determine a bike ride location for a bike ride that the user is participating in. In some embodiments, the assistive system performs the operations in response to a question by the user that does not directly provide instructions to search the user's messages for the bike ride location. In other words, in accordance with some embodiments, the assistive system is capable of performing a set of operations based on a general prompt provided by the user.

As shown in block 406, the head-worn device 120 can automatically begin providing real-time navigation (e.g., via the assistive system or a different navigational application) to the user based on determining that the user is performing a bike ride along a particular navigational route. That is, the assistive system may be capable of determining when a user is performing an activity that can be enhanced by content from a different application stored in memory or otherwise in electronic communication with the head-worn device 120 (e.g., an application stored on the user's smart phone).

As shown in block 408, the head-worn device 120 can provide message readouts from a group message for fellow cyclists to keep the user informed about updates in the chat while the user is performing the physical activity.

As shown in block 410, the head-worn device 120 can provide capabilities for the user to send and receive voice messages to other members of the cycling group chat.

As shown in block 412, the head-worn device 120 can cause the user to receive a text message (e.g., an audio readout of the text message) based on a determination that the message sender is from a user that qualifies under the VIP exceptions for the do not disturb setting that was instantiated at block 402. That is, in some embodiments, the assistive system can determine whether a particular received message should be provided for audio readout to the user based on settings of a different application.

As shown in block 414, the head-worn device 120 can cause a different group thread (e.g., a noisy group thread) to be silenced, such that audio readouts are not provided by the particular messaging thread. As shown in block 416, the assistive system can unmute and catch up on soccer group thread in messenger after ride. As shown in block 418, the assistive system can allow the user to message soccer group thread in messenger in response to a received message from the group thread. As shown in block 420, the assistive system can allow a user to record a voice note about new commitments to soccer group, which may be provided to the user by the assistive system based on a prompt inquiring about the user's availability for a particular event and/or time.

As shown in block 422, the assistive system can allow the user to look up local family events happening this weekend (e.g., by providing a general prompt about the user's availability). In some embodiments, the assistive system can provide the information to the user about the family events based on a different event that has occurred at the head-worn device 120 (e.g., receiving a different message from a different user about the user's availability to participate in a cycling event).

As shown in block 424, the user can receive a summary of a specific family event, for example, in accordance to provide an input in response to receiving the information about local family events happening that weekend. As shown in block 426, the user can provide an input (e.g., “Hey AI assistant, repeat that on my phone”) to cause a previous audio message from the assistive system to be provided at a different electronic device (e.g., “Play last AI response on phone speaker for child to hear”). As shown in block 428, the user can also share feedback from the assistive system (e.g., an AI response) with another user (e.g., the user's partner) on a different application, different than the application that is providing the assistive system (e.g., a messaging application).

As shown in block 430, the user can receive a real-time game notification from sports app. As shown in block 432, the user can cause the assistive system to provide on-demand translation for audio or textual content in another language. In some embodiments, the on-demand translation can be provided automatically based on a user request to read out content that is not in the user's native language. As shown in block 434, the user can request slower speed translation. As shown in block 436, the user can receive voice messages from the cycling group on messenger. As shown in block 438, the user can mute a noisy messenger group chat, which the assistive system may be configured to automatically recognize based on a frequency that electronic messages are being received by the head-worn device 120 or another electronic device in electronic communication with the head-worn device 120. As shown in block 440, the user can check messages.

As shown following block 440, the assistive system can provide a notification to the user about a geographic landmark that the user is in proximity too (e.g., as determined by a navigational application on the user's phone (e.g., “Location Update: At Farmer's Market”). As shown in block 444, the assistive system can be configured to provide new recipe ideas for a new ingredient (e.g., an ingredient purchased at the farmer's market). In some embodiments, the suggestions can be provided in accordance with receiving purchase confirmation at the head-wearable device about a purchase that the user made at the farmers' market.

FIG. 4B illustrates another timeline view of another interaction sequence with a head-worn device 120 (e.g., AR device 700) while a user of the device is performing a sequence of daily activities. As shown in FIG. 4B, the user can engage in an AI chat session (as indicated by the red segment) to perform various activities to start their day (e.g., block 446 to check the local time while traveling, block 448 to set an alarm to leave for the airport later, block 450 to check the weather to decide what to wear, block 452 to check the calendar for a time and/or location of the next event, block 454 to look up local business address and hours, block 456 to message a colleague, and block 458 to listen to news on a podcast). In some embodiments, once a user activates another application that persistently provides audio feedback (e.g., a podcast), the assistive system can be configured to automatically stop the AI chat session.

After stropping the AI chat session, the user can perform a sequence of touch inputs, which may be used to cause the assistive operations to perform various functions, including those related to the audio outputs of the assistive system (e.g., block 460 to receive a text message reply from a colleague, block 462 to replay to the text message, block 464 to resume a podcast, block 466 to book a rideshare to an upcoming event, block 468 to receive a notification about the arrival of the rideshare, block 470 to check status of the rideshare, block 472 to call the rideshare to clarify pickup location, block 474 to listen to a music playlist while chatting, block 476 to receive an alarm to leave for the airport, block 478 to check a status of a flight, block, 480 to receive a reminder to buy a gift before departure of the flight, block 482 to call a partner on a messaging application, and block 484 to listen to meditation for the user's flight anxiety). In some embodiments, the touch inputs provided by the user corresponding to one or more of blocks are based on universal gestures corresponding to universal inputs at the AR device 728, while one or more other blocks may correspond to user inputs provided to contextual input prompts (e.g., in response to an assistive prompt provided by the head-worn device 120).

Thus, as shown in FIGS. 4A and 4B, the systems described herein allow users to interact with an assistive system provided at the head-worn device 120 to allow for increased efficiency and effectiveness of the user's interactions with the head-worn device 120. For example, the assistive system can allow for the user to use the head-worn device 120 as a tool to help level up their efficiencies, including by allowing for multi-tasking and productivity on the go. The assistive systems and devices described herein also allow the user to interact with the assistive system relatively inconspicuously, allowing for them to perform actions without distracting others around them.

### Example Flow Diagrams of an Artificially Intelligent Agent Includes at Wearable Device

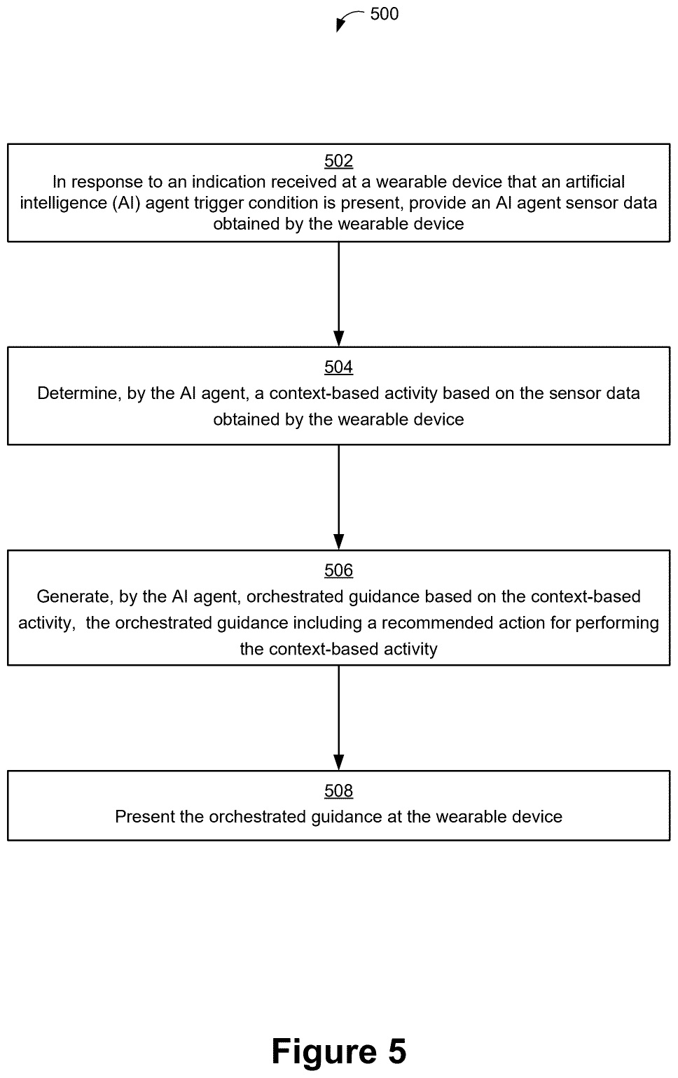

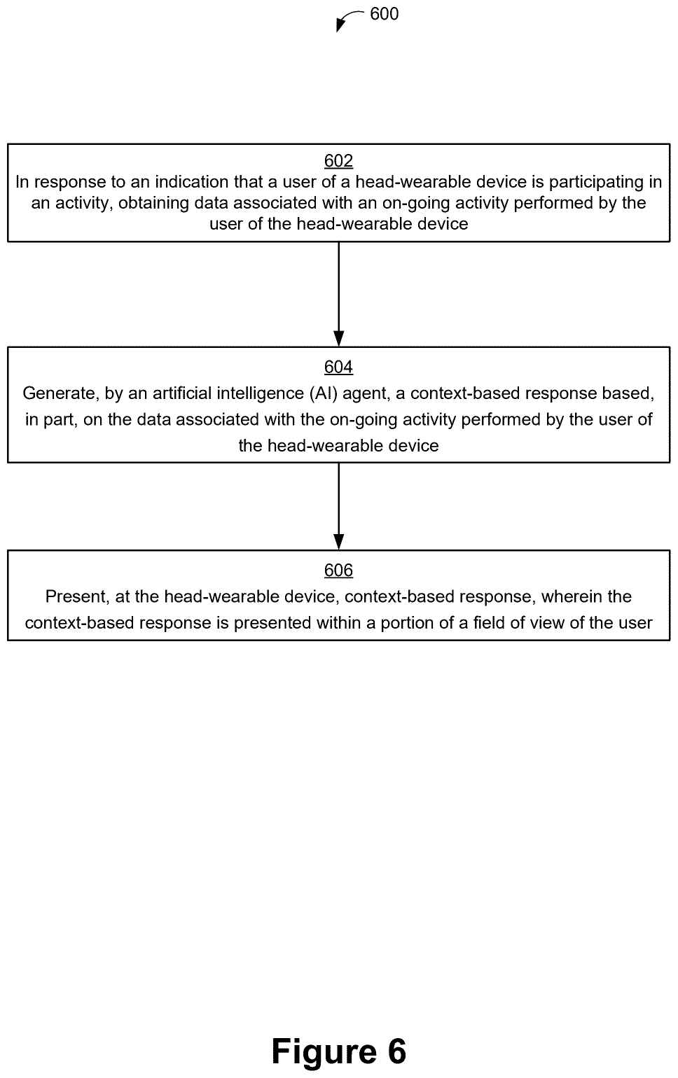

FIGS. 5 and 6 illustrates flow diagrams of methods of generating AI context-based response and actions, in accordance with some embodiments. Operations (e.g., steps) of the methods 500 and 600 can be performed by one or more processors (e.g., central processing unit and/or MCU) of an system XR system (e.g., XR systems of FIGS. 7A-7C-2). At least some of the operations shown in FIGS. 5 and 6 correspond to instructions stored in a computer memory or computer-readable storage medium (e.g., storage, RAM, and/or memory). Operations of the methods 500 and 600 can be performed by a single device alone or in conjunction with one or more processors and/or hardware components of another communicatively coupled device (e.g., wrist-wearable device 110 and a head-wearable device 120) and/or instructions stored in memory or computer-readable medium of the other device communicatively coupled to the system. In some embodiments, the various operations of the methods described herein are interchangeable and/or optional, and respective operations of the methods are performed by any of the aforementioned devices, systems, or combination of devices and/or systems. For convenience, the method operations will be described below as being performed by particular component or device, but should not be construed as limiting the performance of the operation to the particular device in all embodiments.
(A1) FIG. 5 shows a flow chart of a method 500 for generating orchestrated guidance based on an activity of a user, in accordance with some embodiments. The method 500 occurs at a wrist-wearable device 110, head-wearable device 120, and/or other wearable device including one or more sensors, imaging devices, displays, and/or other components described herein. The method 500 includes in response to an indication received at a wearable device that an artificial intelligence (AI) agent trigger condition is present, providing (502) an AI agent sensor data obtained by the wearable device. For example, as shown and described in reference to FIGS. 1A-IN, a wrist-wearable device 110 and/or a head-wearable device 120 of a user can use image data, location data, audio data, and/or other data to detect the presence of an AI agent trigger condition. Non-limiting examples of AI agent trigger conditions include user queries, objects of interest, locations of interest, people of interest, time of day, user invocation, etc.

The method 500 includes determining (504), by the AI agent, a context-based activity based on the sensor data obtained by the wearable device. The context-based activity is an interpretation of a particular activity, action, and/or event with which the user is engaged. For example, as shown and described in reference to FIGS. 1A-IN, the context-based activity is a museum visit or museum tour. Non-limiting examples of context-based activities include shopping, driving, sightseeing, traveling, exploring, cooking, gardening, tours, social meetings, productivity based tasks (e.g., working, note takings, etc.), exercising, etc. The method 500 includes generating (506), by the AI agent, orchestrated guidance based on the context-based activity and presenting (508) the orchestrated guidance at the wearable device.

The orchestrated guidance includes a recommended action for performing the context-based activity. The orchestrated guidance can be a single recommended action, a sequence of recommended actions, and/or or concurrent (and/or parallel) recommended actions for performing the context-based activity. For example, as shown and described in reference to FIGS. 1A-IN, the orchestrated guidance can be one or more recommended actions for facilitating the user's museum tour, such as a recommended action for placing the user devices on “do not disturb,” a recommended action for initiating a guided tour, recommended actions for exploring museum exhibits, presentation of a summary collating missed notifications and/or messages while the user was engaged in the tour, and recommended actions for responding to the missed notifications and/or messages. The orchestrated guidance can be number of recommended actions for assisting the user in performance of the context-based activity—e.g., actions to be performed, during, or after the context-based activity.
(A2) In some embodiments of A1, the context-based activity is a first context-based activity, the sensor data is first sensor data, the orchestrated guidance is first orchestrated guidance, the recommended action is a first recommended action, and the method 500 further includes, in accordance with a determination that the first recommended action for performing the first context-based activity was performed (or was ignored), providing the AI agent second sensor data obtained by the wearable device, determining, by the AI agent, a second context-based activity based on the second sensor data obtained by the wearable device, generating, by the AI agent, second orchestrated guidance based on the second context-based activity and presenting the second orchestrated guidance at the wearable device. The second orchestrated guidance including a second recommended action for performing the second context-based activity. In other words, the method can build on different recommended actions and/or orchestrated guidance. For example, as shown and described in reference to FIGS. 1A-IN, the user can accept one or more recommended actions (e.g., FIGS. 1A-1J) and/or cause the AI agent to generate new recommended actions (e.g., FIGS. 1K-IN-initiating a new context-based activity of searching for a restaurant).
(A3) In some embodiments of any one of A1-A2, the context-based activity is a first context-based activity of a plurality of context-based activities determined by the by the AI agent based on the sensor data, the orchestrated guidance includes a plurality of recommended actions for performing the plurality of context-based activities, and the recommended action is a first recommended action of the plurality of recommended actions, the first recommended action being configured to perform the first context-based activity; and presenting the orchestrated guidance at the wearable device includes presenting at least the first recommended action of the plurality of recommended actions. In other words, any number of context-based activities can be determined for a user and respective orchestrated guidance (and associated recommended actions) can be determined for the context-based activities and presented to the user.
(A4) In some embodiments of A3, generating the orchestrated guidance includes determining a subset of the plurality of recommended actions for performing the first context-based activity, and presenting the orchestrated guidance at the wearable device includes presenting at least the first recommended action of the plurality of recommended actions and the subset of the plurality of recommended actions for performing the first context-based activity. In other words, a plurality recommended of actions associated with a context-based activity can be presented to the user. For example, as shown and described in reference to at least FIG. 1A, at least two recommended actions are presented to the user in accordance with a determination that the user is visiting a museum.
(A5) In some embodiments of any one of A3-A4, generating the orchestrated guidance includes determining a sequence of context-based activities of the plurality of context-based activities to be performed, including a second context-based activity to follow the first context-based activity; and presenting the orchestrated guidance at the wearable device includes presenting at least the first recommended action and the second recommended action of the plurality of recommended actions for performing the plurality of context-based activities. For example, as shown and described in reference to at least FIGS. 1A-1E, a string of recommended actions are presented to the user and the recommended actions are updated based oner inputs selecting one or more user inputs. Similarly, deviations from the recommended actions are shown and described in reference to at least FIGS. 1K-1N.
(A6) In some embodiments of any one of A1-A5, the method 500 includes, in response to a user input selecting the recommended action for performing the context-based activity, causing the wearable device to initiate a do-not-disturb mode (or focus mode, away mode, etc.). While in the do-not-disturb mode, the wearable device suppresses, at least, received notifications, and in response to an indication that participation in the context-based activity ceased causing the wearable device to cease the do-not-disturb mode; generating, by the AI agent, a notification summary based on the notifications received while the wearable device was in the do-not-disturb mode; and presenting the notification summary at the wearable device. Examples of the do-not-disturb mode and the notification summary are shown and described in reference to at least FIGS. 1A-1J.
(A7) In some embodiments of any one of A1-A6, the method 500 includes, in response to a user input selecting the recommended action for performing the context-based activity, performing, by the AI agent, a search based on the recommended action, determining a task to perform based on the search, and presenting the task at the wearable device. An example search request provided by a user is shown and described in reference to at least FIGS. 1K and 1L.
(A8) In some embodiments of any one of A1-A7, presenting the orchestrated guidance at the wearable device includes, at least one of causing presentation of a user interface element associated with the orchestrated guidance at a communicatively coupled display, and causing presentation of audible guidance associated with the orchestrated guidance at a communicatively coupled speaker. An example of one or more user interface elements associated with the orchestrated guidance and audible guidance are shown and described in reference to at least FIG. 1H.
(A9) In some embodiments of any one of A1-A8, the context-based activity is to be performed at a physical activity. For example, as described above, the context-based activity can be an exercise and a recommended action is performance of a particular routine or exercise (detected by the wearable device or another communicatively coupled device).
(B1) In accordance with some embodiments, a method includes receiving sensor data from one or more sensors of a head-wearable device and in response to receiving the data from the one or more sensors of the head-wearable device, processing the data, via an AI agent, to analyze the sensor data to identify a task performed or to be performed by a user, and causing the AI agent to provide guidance associated with performance of the task. For example, a head-wearable device 120 can cause performance of the operations shown and described in reference to FIGS. 1A-IN.
(B2) In some embodiments B1, the causing occurs in response to a selection at a wrist-wearable device of a user interface element that indicates that a guided tour is available. For example as shown and described in reference to FIGS. 1A and 1B, user interface elements corresponding to a guided tour can be presented at a head-wearable device 120 and/or a wrist-wearable device 110.
(B3) In some embodiments of any one of B1 and B2, the sensor data from the one or more sensors is one or more of microphone data, camera data, movement data, and positioning data. In other words, sensor data captured by the wrist-wearable device, the head-wearable device, and/or any other communicatively couple device can be used by the AI agent.
(B4) In some embodiments of any one of B1-B3, the method further includes, after causing the AI agent to provide guidance associated with the task, receiving additional sensor data from the one or more sensors of the head-wearable device, in response to receiving the additional sensor data from the one or more sensors of the head-wearable device, processing the additional sensor data, via the AI agent, to identify an additional task performed or to be performed by the user, and causing the AI agent to provide guidance associated with the additional task. In other words, the AI agent can determine subsequent tasks based on additional data received.
(B5) In some embodiments of B4, the additional task is related to the task.
(C1) In accordance with some embodiments, a method includes receiving a request at an AI agent to (i) forgo immediate output of incoming notifications and (ii) provide a summary of the incoming notifications at a later time, receiving a plurality of notifications, providing the notifications to a large language model (LLM), producing, using the LLM, a summary of the plurality of notifications, and providing a natural language summary, via an output modality of a head-wearable device, at the later time. Examples of summarized notifications are shown and described in reference to FIG. 1J.
(D1) In accordance with some embodiments, a method includes receiving a request from a user interacting with an AI agent, the request requiring traversing content on a website using the AI agent. The method also includes, in response to receiving the request, traversing, using an computer-implemented agent associated with the AI agent, one or more graphical user interfaces associated with the website to collect data needed to formulate a response to the request from the user, and after the traversing, processing the data collected by the computer-implemented agent associated with the AI agent to generate the response and providing the response to the user. For example, as shown and described in reference to FIG. 1K-IN, the AI agent can utilize a web agent to search webpages and/or perform a web search to complete a user request and provide a corresponding response. In some embodiments, the web-based AI agent is distinct from the AI agent that received the task request. In some embodiments, different training data used to train that AI agent and the web-based agent. In some embodiments, the traversing the one or more web pages includes obtaining data needed to formulate a response to the request from the user. In some embodiments, surface UI element related to progress of the AI agent in performing the traversal is presented (e.g., an AI agent symbol moving or spinning to show progress). In some embodiment, the web-based agent can be used to inquire about a contact (e.g., ask about a particular person that may be a contact of the user—e.g., “What kind of trip would Mike go on?”).

In some embodiments of A1-D1, the context-based activities are further determined based on stored user data (e.g., use data about the user's previous experiences and/or interests to curate the information about the guided tour). For example, if the user previously participated in an experience that was relevant to an aspect of the guided tour (e.g., FIGS. 1A-IN), the AI agent may cause information about the previous event to surface or otherwise be integrated into the guided tour.

In some embodiments of A1-D1, a head-wearable device is a display-less AR headset. In some embodiments, the input/output interface of the head-wearable device only includes one or more speakers. In some embodiments, the operations of the head-wearable device can be performed by a set of earbuds or other head-worn speaker device.)

In some embodiments of A1-D1, a user interface associated with the orchestrated set of guidance instructions is provided by the AI agent via a Lo-Fi display, the Lo-Fi being a glanceable display that presents notifications, live activities, AI agent information, and messages.

In some embodiments of A1-D1, a user interface associated with the orchestrated set of guidance instructions is provided by the AI agent via a projector display, the projector display configured to project information at a hand of the user (e.g., at a palm or other body part of the user).

In some embodiments of A1-D1, a non-textual user interface element is presented at the head-wearable device (e.g., an audio message, an arrow or similar symbol), and the non-textual user interface element is configured to direct a user of the head-wearable device toward a physical landmark as part of the orchestrated set of guidance instructions.

In some embodiments of A1-D1, the user can select objects within a field of view of the user (e.g., captured by one or more sensors of a wearable device, such as an imaging device) to receive additional information on the selected object.

In some embodiments of A1-D1, the AI agent may cause some notifications to be muted during the guided tour, and then provide with an AI-generated summary of the conversations later so that the user can quickly catch up without reviewing many different messages right away.
(E1) FIG. 6 shows a flow chart of a method 600 for facilitating performance of a physical activity performed by user, in accordance with some embodiments. The method 600 occurs at a wrist-wearable device 110, a head-wearable device 120, and/or other wearable device including one or more sensors, imaging devices, displays, and/or other components described herein. The method 600 includes, in response to an indication that a user of a head-wearable device is participating in an activity, obtaining (602) data associated with an on-going activity performed by the user of the head-wearable device. The method 600 includes generating (604), by an AI agent, a context-based response based, in part, on the data associated with the on-going activity performed by the user of the head-wearable device, and presenting (606), at the head-wearable device, context-based response. The context-based response is presented within a portion of a field of view of the user. For example, as shown and described in reference to FIGS. 2A-2H, a head-wearable device 120 can present different context-based responses to the user based on a physical activity being performed.
(E2) In some embodiments of E1, the method 600 includes, in accordance with a determination that the activity is a group activity performed with at least one contact of the user, obtaining, from an electronic device associated with the at least one contact of the user, additional data associated with a respective on-going activity performed by the at least one contact of the user. The context-based response is further based on the additional data associated with the respective on-going activity performed by the at least one contact of the user. For example, as shown and described in reference to FIGS. 2I-2R, an AI agent can detect other contacts performing an activity with a user and share information between the users.
(E3) In some embodiments of E2, the data associated with the on-going activity performed by the user of the head-wearable device and the additional data associated with the respective on-going activity performed by the at least one contact of the user includes respective image data and/or audio data, and the context-based response is an image response including a combination of the respective image data. For example, as shown and described in reference to FIG. 2L, image data captured between the wearable devices can be synchronized, combined into a single image, and/or combined into an image collage.
(E4) In some embodiments of E3, the respective image data includes a plurality of video streams from a plurality of respective head-wearable devices, and generating, by the AI agent, the context-based response includes in accordance with a determination that the plurality of video streams are (i) captured within a predefined amount of time of each other and (ii) within a predefined distance of each other, preparing a collated video of two or more of the plurality of video streams in a time-synchronized fashion. In some embodiments, the method includes providing to each of the respective head-wearable devices the collated video. At least one aspect of the collated video provided to each of the respective head-wearable devices is tailored to that respective head-wearable device.
(E5) In some embodiments of any one of E1-E4, the activity is a physical exercise; and the context-based response is a coaching response to assist the user on performance of the physical exercise. For example, as shown and described in reference to FIGS. 2A-2H, an AI agent can coach a user through an exercise.
(E6) In some embodiments of any one of E1-E5, the activity is outdoor physical activity (e.g., running, biking, hiking, etc.), and the context-based response is a navigation instructions. For example, as shown in at least FIGS. 3A and 3B, the AI agent can provide navigation instructions to the user.
(E7) In some embodiments of any one of E1-E6, the activity is participation in a note-taking session (e.g., a meeting, class, lecture, etc.), and the context-based response is a request to generate notes. While the primary example shown in FIGS. 2A-2R is an exercise, the AI agent can be used with other activities performed by the user.
(F1) In accordance with some embodiments, a method is performed at a head-wearable device including (i) one or more cameras, and (ii) a display component configured to display digital content. The method includes determining that a user wearing the head-wearable device is performing a physical activity and, in accordance with determining that the user wearing the head-wearable device is performing the physical activity, automatically, without additional user input, initializing assistive operations based on data provided by the one or more cameras of the head-wearable device. The method also includes, while the assistive operations are being performed based on image data from the one or more cameras of the head-wearable device, identifying, based on the assistive operations, that at least a portion of a respective field of view of a respective camera of the one or more cameras satisfies automatic-image-capture criteria for automatically capturing an image. The method further includes, based on the identifying, causing the respective camera to capture an image automatically, without further user input. For example, as shown and described in reference to FIG. 3A, a wearable device can automatically capture image data.
(F2) In some embodiments for F1, the method further includes detecting a user input directed to a universal action button on a peripheral portion of the head-wearable device. The assistive operations are initialized based on the user input being detected while the user is performing the physical activity. For example, as shown and described in reference to FIG. 2A, the user can perform a tap gesture at a wearable device, such as the head-wearable device, to initiate the AI agent and/or other operations.
(G1) In accordance with some embodiments, a method includes receiving (i) performance data corresponding to a physical activity that a user of a head-wearable device is performing, and (ii) capturing image data by the head-wearable device during performance of the physical activity. The method also includes causing presentation, at a display component of the head-wearable device, a user interface element that includes one or more representations of the performance data, and responsive to provided user preferences, automatically sharing a field of view of the user in conjunction with sharing the user interface element as a composite user interface element to one or more other electronic devices. For example, as shown and described in reference to FIGS. 2G-2L, information captured by wearable devices can be shared between users.
(G2) In some embodiments of G1, the performance data is received from a software application different than another software application that is performing operations at the head-wearable device for capturing the image data. For example, the information can be received from a streaming application and/or other application.
(H1) In accordance with some embodiments, a method includes determining that a user of a head-wearable device is beginning performance of a physical activity while data about the physical activity is configured to be obtained by the head-wearable device of the user and, in accordance with the determining that the user of the head-wearable device is beginning performance of the physical activity, identifying an assistive module that uses one or more specialized artificial-intelligence models. The method also includes causing interactive content to be provided to the user via the assistive module based on the data obtained about the physical activity that the user is performing. For example, as shown and described in reference to FIGS. 2A-2D, information captured by wearable devices can be used to assist the user in performance of the activity.
(H2) In some embodiments of H1, the method further includes generating an audio message using an artificial intelligence model of the assistive module performing operations during performance of the physical activity by the user and determining based on data obtained about performance of the physical activity by the user, that one or more message-providing criteria are satisfied. The method also includes, in accordance with the determining that the one or more message-providing criteria are satisfied, generating, using an AI model, a message related to the performance of the physical activity, and providing the generated electronic message to the user via one or of (i) a microphone of the head-wearable device, and (ii) a display component within a frame of the head-wearable device.
(I1) In accordance with some embodiments, a method includes, at a head-worn device including a user interface for providing user interface elements to a user based on physical activities that the user is performing, receiving an update about a location of a user, based on a physical activity that the user is performing, and in accordance with receiving the indication, presenting a navigational user interface to the user providing navigation to the user based on an identified activity that the user is performing while wearing a head-worn device. For example, as shown and described above in reference to FIG. 3A, navigation instructions can be provided to the user.
(J1) In accordance with some embodiments, a method of providing personalized physical activity notifications is disclosed. The method is performed by a system including one or more processors communicatively coupled with a head-wearable device and one or more sensors. The method includes, while a user wearing a head-wearable device is performing a physical activity, obtaining sensor data sensed from the user via one or more sensors. The sensor data includes at least biometric data and physical activity data. The method includes, in response to a user input querying the physical activity, initiating an artificial intelligence (AI) agent and generating, by the AI agent, an AI message responsive to the user input. The AI message is based on one or more of the sensor data and the physical activity data. The method also includes causing presentation of the AI message via the head-wearable device. Examples of one or more AI messages generated by an AI agent while a user wearing, at least, a head-wearable device are shown and describe above in reference to FIGS. 2A-3D.
(J2) In some embodiments of J1, the method includes receiving additional sensor data from one or more of first sensors of an electronic device communicatively coupled with the head-wearable device, second sensors of a wrist-wearable device with the head-wearable device and/or the electronic device, and one or more programs operating on one or more of the electronic device, the head-wearable device, the wrist-wearable device, and the system. The method further includes, before generating the AI message responsive to the user input, updating the sensor data based on the additional sensor data. As described above in reference to FIGS. 2A-3D, the AI agent can use any device of an XR systems (FIGS. 7A-7C) to obtain data used for generating AI messages and/or responses.
(J3) In some embodiments of any of J1-J2, the method includes, while the user is performing a physical activity, in accordance with a determination that one or more capture triggers are satisfied, capturing image data via one or more imaging devices of the head-wearable device. The method also includes, in response to an indication that the user has ceased performing the physical activity, generating, by the AI agent, a physical activity summary based on the sensor data and the image data. The physical activity summary includes a compilation of image data during performance of the physical activity, an overview of the physical activity, and personal records. The method also includes causing an electronic device communicatively coupled with the head-wearable device to present the physical activity summary. Examples of AI messages, including physical activity summaries, are shown and described above in reference to FIGS. 2A-3D.
(J4) In some embodiments of any of J1-J3, the user input querying the physical activity is a progress update. The AI message includes a real-time progress update for the physical activity including one or more of a distance, an elapsed time, a heart rate, an elevation, a location, power zones, a pace, a speed, split information, a cadence, and fitness application information. Examples of A1 messages are shown and described above in reference to FIGS. 2A-3D.
(J5) In some embodiments of any of J1-J4, the user input querying the physical activity is an in-activity insight request. The AI message includes real-time metric data, a physical activity summary, physical activity progress, user target progress, and historical performance information. Examples of AI messages are shown and described above in reference to FIGS. 2A-3D.
(J6) In some embodiments of any of J1-J4, the method further includes, in accordance with a determination that one or more progress triggers are satisfied, generating, by the AI agent, physical activity notification including one or more of real-time metrics, personal goal progress, and physical activity progress. The method also includes causing presentation of the physical activity notification via the head-wearable device. Examples of progress triggers and physical activity notifications are shown and described above in reference to FIGS. 2A-3D.
(J7) In some embodiments of any of J1-J6, the one or more progress triggers include one or more of an event trigger, a predetermined threshold, and a predetermined interval.
(K1) In accordance with some embodiments, a system that includes one or more wrist wearable devices and a pair of augmented-reality glasses, and the system is configured to perform operations corresponding to any of A1-J7.
(L1) In accordance with some embodiments, a non-transitory computer readable storage medium including instructions that, when executed by a computing device in communication with a pair of augmented-reality glasses, cause the computer device to perform operations corresponding to any of A1-I1.
(M1) In accordance with some embodiments, a means for performing or causing performance of operations corresponding to any of A1-I1.
(N1) In accordance with some embodiments, a wearable device (a head-wearable device and/or a wrist-wearable device) configured to perform or cause performance of operations corresponding to any of A1-I1.
(O1) In accordance with some embodiments, an intermediary processing device (e.g., configured to offload processing operations for a wrist-wearable device and/or a head-worn device (e.g. augmented-reality glasses)) configured to perform or cause performance operations corresponding to any of A1-I1.

### Example Extended-Reality Systems

FIGS. 7A-7C-2 illustrate example XR systems that include AR and MR systems, in accordance with some embodiments. FIG. 7A shows a first XR system 700a and first example user interactions using a wrist-wearable device 726, a head-wearable device (e.g., AR device 728), and/or a HIPD 742. FIG. 7B shows a second XR system 700b and second example user interactions using a wrist-wearable device 726, AR device 728, and/or an HIPD 742. FIGS. 7C-1 and 7C-2 show a third MR system 700c and third example user interactions using a wrist-wearable device 726, a head-wearable device (e.g., an MR device such as a VR device), and/or an HIPD 742. As the skilled artisan will appreciate upon reading the descriptions provided herein, the above-example AR and MR systems (described in detail below) can perform various functions and/or operations.

The wrist-wearable device 726, the head-wearable devices, and/or the HIPD 742 can communicatively couple via a network 725 (e.g., cellular, near field, Wi-Fi, personal area network, wireless LAN). Additionally, the wrist-wearable device 726, the head-wearable device, and/or the HIPD 742 can also communicatively couple with one or more servers 730, computers 740 (e.g., laptops, computers), mobile devices 750 (e.g., smartphones, tablets), and/or other electronic devices via the network 725 (e.g., cellular, near field, Wi-Fi, personal area network, wireless LAN). Similarly, a smart textile-based garment, when used, can also communicatively couple with the wrist-wearable device 726, the head-wearable device(s), the HIPD 742, the one or more servers 730, the computers 740, the mobile devices 750, and/or other electronic devices via the network 725 to provide inputs.

Turning to FIG. 7A, a user 702 is shown wearing the wrist-wearable device 726 and the AR device 728 and having the HIPD 742 on their desk. The wrist-wearable device 726, the AR device 728, and the HIPD 742 facilitate user interaction with an AR environment. In particular, as shown by the first AR system 700a, the wrist-wearable device 726, the AR device 728, and/or the HIPD 742 cause presentation of one or more avatars 704, digital representations of contacts 706, and virtual objects 708. As discussed below, the user 702 can interact with the one or more avatars 704, digital representations of the contacts 706, and virtual objects 708 via the wrist-wearable device 726, the AR device 728, and/or the HIPD 742. In addition, the user 702 is also able to directly view physical objects in the environment, such as a physical table 729, through transparent lens(es) and waveguide(s) of the AR device 728. Alternatively, an MR device could be used in place of the AR device 728 and a similar user experience can take place, but the user would not be directly viewing physical objects in the environment, such as table 729, and would instead be presented with a virtual reconstruction of the table 729 produced from one or more sensors of the MR device (e.g., an outward facing camera capable of recording the surrounding environment).

The user 702 can use any of the wrist-wearable device 726, the AR device 728 (e.g., through physical inputs at the AR device and/or built-in motion tracking of a user's extremities), a smart-textile garment, externally mounted extremity tracking device, the HIPD 742 to provide user inputs, etc. For example, the user 702 can perform one or more hand gestures that are detected by the wrist-wearable device 726 (e.g., using one or more EMG sensors and/or IMUs built into the wrist-wearable device) and/or AR device 728 (e.g., using one or more image sensors or cameras) to provide a user input. Alternatively, or additionally, the user 702 can provide a user input via one or more touch surfaces of the wrist-wearable device 726, the AR device 728, and/or the HIPD 742, and/or voice commands captured by a microphone of the wrist-wearable device 726, the AR device 728, and/or the HIPD 742. The wrist-wearable device 726, the AR device 728, and/or the HIPD 742 include an artificially intelligent digital assistant to help the user in providing a user input (e.g., completing a sequence of operations, suggesting different operations or commands, providing reminders, confirming a command). For example, the digital assistant can be invoked through an input occurring at the AR device 728 (e.g., via an input at a temple arm of the AR device 728). In some embodiments, the user 702 can provide a user input via one or more facial gestures and/or facial expressions. For example, cameras of the wrist-wearable device 726, the AR device 728, and/or the HIPD 742 can track the user 702's eyes for navigating a user interface.

The wrist-wearable device 726, the AR device 728, and/or the HIPD 742 can operate alone or in conjunction to allow the user 702 to interact with the AR environment. In some embodiments, the HIPD 742 is configured to operate as a central hub or control center for the wrist-wearable device 726, the AR device 728, and/or another communicatively coupled device. For example, the user 702 can provide an input to interact with the AR environment at any of the wrist-wearable device 726, the AR device 728, and/or the HIPD 742, and the HIPD 742 can identify one or more back-end and front-end tasks to cause the performance of the requested interaction and distribute instructions to cause the performance of the one or more back-end and front-end tasks at the wrist-wearable device 726, the AR device 728, and/or the HIPD 742. In some embodiments, a back-end task is a background-processing task that is not perceptible by the user (e.g., rendering content, decompression, compression, application-specific operations), and a front-end task is a user-facing task that is perceptible to the user (e.g., presenting information to the user, providing feedback to the user). The HIPD 742 can perform the back-end tasks and provide the wrist-wearable device 726 and/or the AR device 728 operational data corresponding to the performed back-end tasks such that the wrist-wearable device 726 and/or the AR device 728 can perform the front-end tasks. In this way, the HIPD 742, which has more computational resources and greater thermal headroom than the wrist-wearable device 726 and/or the AR device 728, performs computationally intensive tasks and reduces the computer resource utilization and/or power usage of the wrist-wearable device 726 and/or the AR device 728.

In the example shown by the first AR system 700a, the HIPD 742 identifies one or more back-end tasks and front-end tasks associated with a user request to initiate an AR video call with one or more other users (represented by the avatar 704 and the digital representation of the contact 706) and distributes instructions to cause the performance of the one or more back-end tasks and front-end tasks. In particular, the HIPD 742 performs back-end tasks for processing and/or rendering image data (and other data) associated with the AR video call and provides operational data associated with the performed back-end tasks to the AR device 728 such that the AR device 728 performs front-end tasks for presenting the AR video call (e.g., presenting the avatar 704 and the digital representation of the contact 706).

In some embodiments, the HIPD 742 can operate as a focal or anchor point for causing the presentation of information. This allows the user 702 to be generally aware of where information is presented. For example, as shown in the first AR system 700a, the avatar 704 and the digital representation of the contact 706 are presented above the HIPD 742. In particular, the HIPD 742 and the AR device 728 operate in conjunction to determine a location for presenting the avatar 704 and the digital representation of the contact 706. In some embodiments, information can be presented within a predetermined distance from the HIPD 742 (e.g., within five meters). For example, as shown in the first AR system 700a, virtual object 708 is presented on the desk some distance from the HIPD 742. Similar to the above example, the HIPD 742 and the AR device 728 can operate in conjunction to determine a location for presenting the virtual object 708. Alternatively, in some embodiments, presentation of information is not bound by the HIPD 742. More specifically, the avatar 704, the digital representation of the contact 706, and the virtual object 708 do not have to be presented within a predetermined distance of the HIPD 742. While an AR device 728 is described working with an HIPD, an MR headset can be interacted with in the same way as the AR device 728.

User inputs provided at the wrist-wearable device 726, the AR device 728, and/or the HIPD 742 are coordinated such that the user can use any device to initiate, continue, and/or complete an operation. For example, the user 702 can provide a user input to the AR device 728 to cause the AR device 728 to present the virtual object 708 and, while the virtual object 708 is presented by the AR device 728, the user 702 can provide one or more hand gestures via the wrist-wearable device 726 to interact and/or manipulate the virtual object 708. While an AR device 728 is described working with a wrist-wearable device 726, an MR headset can be interacted with in the same way as the AR device 728.

Integration of Artificial Intelligence with XR Systems

FIG. 7A illustrates an interaction in which an artificially intelligent virtual assistant can assist in requests made by a user 702. The AI virtual assistant can be used to complete open-ended requests made through natural language inputs by a user 702. For example, in FIG. 7A the user 702 makes an audible request 744 to summarize the conversation and then share the summarized conversation with others in the meeting. In addition, the AI virtual assistant is configured to use sensors of the XR system (e.g., cameras of an XR headset, microphones, and various other sensors of any of the devices in the system) to provide contextual prompts to the user for initiating tasks.

FIG. 7A also illustrates an example neural network 752 used in Artificial Intelligence applications. Uses of Artificial Intelligence (AI) are varied and encompass many different aspects of the devices and systems described herein. AI capabilities cover a diverse range of applications and deepen interactions between the user 702 and user devices (e.g., the AR device 728, an MR device 732, the HIPD 742, the wrist-wearable device 726). The AI discussed herein can be derived using many different training techniques. While the primary AI model example discussed herein is a neural network, other AI models can be used. Non-limiting examples of AI models include artificial neural networks (ANNs), deep neural networks (DNNs), convolution neural networks (CNNs), recurrent neural networks (RNNs), large language models (LLMs), long short-term memory networks, transformer models, decision trees, random forests, support vector machines, k-nearest neighbors, genetic algorithms, Markov models, Bayesian networks, fuzzy logic systems, and deep reinforcement learnings, etc. The AI models can be implemented at one or more of the user devices, and/or any other devices described herein. For devices and systems herein that employ multiple AI models, different models can be used depending on the task. For example, for a natural-language artificially intelligent virtual assistant, an LLM can be used and for the object detection of a physical environment, a DNN can be used instead.

In another example, an AI virtual assistant can include many different AI models and based on the user's request, multiple AI models may be employed (concurrently, sequentially or a combination thereof). For example, an LLM-based AI model can provide instructions for helping a user follow a recipe and the instructions can be based in part on another AI model that is derived from an ANN, a DNN, an RNN, etc. that is capable of discerning what part of the recipe the user is on (e.g., object and scene detection).

As AI training models evolve, the operations and experiences described herein could potentially be performed with different models other than those listed above, and a person skilled in the art would understand that the list above is non-limiting.

A user 702 can interact with an AI model through natural language inputs captured by a voice sensor, text inputs, or any other input modality that accepts natural language and/or a corresponding voice sensor module. In another instance, input is provided by tracking the eye gaze of a user 702 via a gaze tracker module. Additionally, the AI model can also receive inputs beyond those supplied by a user 702. For example, the AI can generate its response further based on environmental inputs (e.g., temperature data, image data, video data, ambient light data, audio data, GPS location data, inertial measurement (i.e., user motion) data, pattern recognition data, magnetometer data, depth data, pressure data, force data, neuromuscular data, heart rate data, temperature data, sleep data) captured in response to a user request by various types of sensors and/or their corresponding sensor modules. The sensors' data can be retrieved entirely from a single device (e.g., AR device 728) or from multiple devices that are in communication with each other (e.g., a system that includes at least two of an AR device 728, an MR device 732, the HIPD 742, the wrist-wearable device 726, etc.). The AI model can also access additional information (e.g., one or more servers 730, the computers 740, the mobile devices 750, and/or other electronic devices) via a network 725.

A non-limiting list of AI-enhanced functions includes but is not limited to image recognition, speech recognition (e.g., automatic speech recognition), text recognition (e.g., scene text recognition), pattern recognition, natural language processing and understanding, classification, regression, clustering, anomaly detection, sequence generation, content generation, and optimization. In some embodiments, AI-enhanced functions are fully or partially executed on cloud-computing platforms communicatively coupled to the user devices (e.g., the AR device 728, an MR device 732, the HIPD 742, the wrist-wearable device 726) via the one or more networks. The cloud-computing platforms provide scalable computing resources, distributed computing, managed AI services, interference acceleration, pre-trained models, APIs and/or other resources to support comprehensive computations required by the AI-enhanced function.

Example outputs stemming from the use of an AI model can include natural language responses, mathematical calculations, charts displaying information, audio, images, videos, texts, summaries of meetings, predictive operations based on environmental factors, classifications, pattern recognitions, recommendations, assessments, or other operations. In some embodiments, the generated outputs are stored on local memories of the user devices (e.g., the AR device 728, an MR device 732, the HIPD 742, the wrist-wearable device 726), storage options of the external devices (servers, computers, mobile devices, etc.), and/or storage options of the cloud-computing platforms.

The AI-based outputs can be presented across different modalities (e.g., audio-based, visual-based, haptic-based, and any combination thereof) and across different devices of the XR system described herein. Some visual-based outputs can include the displaying of information on XR augments of an XR headset, user interfaces displayed at a wrist-wearable device, laptop device, mobile device, etc. On devices with or without displays (e.g., HIPD 742), haptic feedback can provide information to the user 702. An AI model can also use the inputs described above to determine the appropriate modality and device(s) to present content to the user (e.g., a user walking on a busy road can be presented with an audio output instead of a visual output to avoid distracting the user 702).

### Example Augmented Reality Interaction

FIG. 7B shows the user 702 wearing the wrist-wearable device 726 and the AR device 728 and holding the HIPD 742. In the second AR system 700b, the wrist-wearable device 726, the AR device 728, and/or the HIPD 742 are used to receive and/or provide one or more messages to a contact of the user 702. In particular, the wrist-wearable device 726, the AR device 728, and/or the HIPD 742 detect and coordinate one or more user inputs to initiate a messaging application and prepare a response to a received message via the messaging application.

In some embodiments, the user 702 initiates, via a user input, an application on the wrist-wearable device 726, the AR device 728, and/or the HIPD 742 that causes the application to initiate on at least one device. For example, in the second AR system 700b the user 702 performs a hand gesture associated with a command for initiating a messaging application (represented by messaging user interface 712); the wrist-wearable device 726 detects the hand gesture; and, based on a determination that the user 702 is wearing the AR device 728, causes the AR device 728 to present a messaging user interface 712 of the messaging application. The AR device 728 can present the messaging user interface 712 to the user 702 via its display (e.g., as shown by user 702's field of view 710). In some embodiments, the application is initiated and can be run on the device (e.g., the wrist-wearable device 726, the AR device 728, and/or the HIPD 742) that detects the user input to initiate the application, and the device provides another device operational data to cause the presentation of the messaging application. For example, the wrist-wearable device 726 can detect the user input to initiate a messaging application, initiate and run the messaging application, and provide operational data to the AR device 728 and/or the HIPD 742 to cause presentation of the messaging application. Alternatively, the application can be initiated and run at a device other than the device that detected the user input. For example, the wrist-wearable device 726 can detect the hand gesture associated with initiating the messaging application and cause the HIPD 742 to run the messaging application and coordinate the presentation of the messaging application.

Further, the user 702 can provide a user input provided at the wrist-wearable device 726, the AR device 728, and/or the HIPD 742 to continue and/or complete an operation initiated at another device. For example, after initiating the messaging application via the wrist-wearable device 726 and while the AR device 728 presents the messaging user interface 712, the user 702 can provide an input at the HIPD 742 to prepare a response (e.g., shown by the swipe gesture performed on the HIPD 742). The user 702's gestures performed on the HIPD 742 can be provided and/or displayed on another device. For example, the user 702's swipe gestures performed on the HIPD 742 are displayed on a virtual keyboard of the messaging user interface 712 displayed by the AR device 728.

In some embodiments, the wrist-wearable device 726, the AR device 728, the HIPD 742, and/or other communicatively coupled devices can present one or more notifications to the user 702. The notification can be an indication of a new message, an incoming call, an application update, a status update, etc. The user 702 can select the notification via the wrist-wearable device 726, the AR device 728, or the HIPD 742 and cause presentation of an application or operation associated with the notification on at least one device. For example, the user 702 can receive a notification that a message was received at the wrist-wearable device 726, the AR device 728, the HIPD 742, and/or other communicatively coupled device and provide a user input at the wrist-wearable device 726, the AR device 728, and/or the HIPD 742 to review the notification, and the device detecting the user input can cause an application associated with the notification to be initiated and/or presented at the wrist-wearable device 726, the AR device 728, and/or the HIPD 742.

While the above example describes coordinated inputs used to interact with a messaging application, the skilled artisan will appreciate upon reading the descriptions that user inputs can be coordinated to interact with any number of applications including, but not limited to, gaming applications, social media applications, camera applications, web-based applications, financial applications, etc. For example, the AR device 728 can present to the user 702 game application data and the HIPD 742 can use a controller to provide inputs to the game. Similarly, the user 702 can use the wrist-wearable device 726 to initiate a camera of the AR device 728, and the user can use the wrist-wearable device 726, the AR device 728, and/or the HIPD 742 to manipulate the image capture (e.g., zoom in or out, apply filters) and capture image data.

While an AR device 728 is shown being capable of certain functions, it is understood that an AR device can be an AR device with varying functionalities based on costs and market demands. For example, an AR device may include a single output modality such as an audio output modality. In another example, the AR device may include a low-fidelity display as one of the output modalities, where simple information (e.g., text and/or low-fidelity images/video) is capable of being presented to the user. In yet another example, the AR device can be configured with face-facing light emitting diodes (LEDs) configured to provide a user with information, e.g., an LED around the right-side lens can illuminate to notify the wearer to turn right while directions are being provided or an LED on the left-side can illuminate to notify the wearer to turn left while directions are being provided. In another embodiment, the AR device can include an outward-facing projector such that information (e.g., text information, media) may be displayed on the palm of a user's hand or other suitable surface (e.g., a table, whiteboard). In yet another embodiment, information may also be provided by locally dimming portions of a lens to emphasize portions of the environment in which the user's attention should be directed. Some AR devices can present AR augments either monocularly or binocularly (e.g., an AR augment can be presented at only a single display associated with a single lens as opposed presenting an AR augmented at both lenses to produce a binocular image). In some instances an AR device capable of presenting AR augments binocularly can optionally display AR augments monocularly as well (e.g., for power-saving purposes or other presentation considerations). These examples are non-exhaustive and features of one AR device described above can be combined with features of another AR device described above. While features and experiences of an AR device have been described generally in the preceding sections, it is understood that the described functionalities and experiences can be applied in a similar manner to an MR headset, which is described below in the proceeding sections.

### Example Mixed Reality Interaction

Turning to FIGS. 7C-1 and 7C-2, the user 702 is shown wearing the wrist-wearable device 726 and an MR device 732 (e.g., a device capable of providing either an entirely VR experience or an MR experience that displays object(s) from a physical environment at a display of the device) and holding the HIPD 742. In the third AR system 700c, the wrist-wearable device 726, the MR device 732, and/or the HIPD 742 are used to interact within an MR environment, such as a VR game or other MR/VR application. While the MR device 732 presents a representation of a VR game (e.g., first MR game environment 720) to the user 702, the wrist-wearable device 726, the MR device 732, and/or the HIPD 742 detect and coordinate one or more user inputs to allow the user 702 to interact with the VR game.

In some embodiments, the user 702 can provide a user input via the wrist-wearable device 726, the MR device 732, and/or the HIPD 742 that causes an action in a corresponding MR environment. For example, the user 702 in the third MR system 700c (shown in FIG. 7C-1) raises the HIPD 742 to prepare for a swing in the first MR game environment 720. The MR device 732, responsive to the user 702 raising the HIPD 742, causes the MR representation of the user 722 to perform a similar action (e.g., raise a virtual object, such as a virtual sword 724). In some embodiments, each device uses respective sensor data and/or image data to detect the user input and provide an accurate representation of the user 702's motion. For example, image sensors (e.g., SLAM cameras or other cameras) of the HIPD 742 can be used to detect a position of the HIPD 742 relative to the user 702's body such that the virtual object can be positioned appropriately within the first MR game environment 720; sensor data from the wrist-wearable device 726 can be used to detect a velocity at which the user 702 raises the HIPD 742 such that the MR representation of the user 722 and the virtual sword 724 are synchronized with the user 702's movements; and image sensors of the MR device 732 can be used to represent the user 702's body, boundary conditions, or real-world objects within the first MR game environment 720.

In FIG. 7C-2, the user 702 performs a downward swing while holding the HIPD 742. The user 702's downward swing is detected by the wrist-wearable device 726, the MR device 732, and/or the HIPD 742 and a corresponding action is performed in the first MR game environment 720. In some embodiments, the data captured by each device is used to improve the user's experience within the MR environment. For example, sensor data of the wrist-wearable device 726 can be used to determine a speed and/or force at which the downward swing is performed and image sensors of the HIPD 742 and/or the MR device 732 can be used to determine a location of the swing and how it should be represented in the first MR game environment 720, which, in turn, can be used as inputs for the MR environment (e.g., game mechanics, which can use detected speed, force, locations, and/or aspects of the user 702's actions to classify a user's inputs (e.g., user performs a light strike, hard strike, critical strike, glancing strike, miss) or calculate an output (e.g., amount of damage)).

FIG. 7C-2 further illustrates that a portion of the physical environment is reconstructed and displayed at a display of the MR device 732 while the MR game environment 720 is being displayed. In this instance, a reconstruction of the physical environment 746 is displayed in place of a portion of the MR game environment 720 when object(s) in the physical environment are potentially in the path of the user (e.g., a collision with the user and an object in the physical environment are likely). Thus, this example MR game environment 720 includes (i) an immersive VR portion 748 (e.g., an environment that does not have a corollary counterpart in a nearby physical environment) and (ii) a reconstruction of the physical environment 746 (e.g., table 750 and cup 752). While the example shown here is an MR environment that shows a reconstruction of the physical environment to avoid collisions, other uses of reconstructions of the physical environment can be used, such as defining features of the virtual environment based on the surrounding physical environment (e.g., a virtual column can be placed based on an object in the surrounding physical environment (e.g., a tree)).

While the wrist-wearable device 726, the MR device 732, and/or the HIPD 742 are described as detecting user inputs, in some embodiments, user inputs are detected at a single device (with the single device being responsible for distributing signals to the other devices for performing the user input). For example, the HIPD 742 can operate an application for generating the first MR game environment 720 and provide the MR device 732 with corresponding data for causing the presentation of the first MR game environment 720, as well as detect the user 702's movements (while holding the HIPD 742) to cause the performance of corresponding actions within the first MR game environment 720. Additionally or alternatively, in some embodiments, operational data (e.g., sensor data, image data, application data, device data, and/or other data) of one or more devices is provided to a single device (e.g., the HIPD 742) to process the operational data and cause respective devices to perform an action associated with processed operational data.

In some embodiments, the user 702 can wear a wrist-wearable device 726, wear an MR device 732, wear smart textile-based garments 738 (e.g., wearable haptic gloves), and/or hold an HIPD 742 device. In this embodiment, the wrist-wearable device 726, the MR device 732, and/or the smart textile-based garments 738 are used to interact within an MR environment (e.g., any AR or MR system described above in reference to FIGS. 7A-7B). While the MR device 732 presents a representation of an MR game (e.g., second MR game environment 720) to the user 702, the wrist-wearable device 726, the MR device 732, and/or the smart textile-based garments 738 detect and coordinate one or more user inputs to allow the user 702 to interact with the MR environment.

In some embodiments, the user 702 can provide a user input via the wrist-wearable device 726, an HIPD 742, the MR device 732, and/or the smart textile-based garments 738 that causes an action in a corresponding MR environment. In some embodiments, each device uses respective sensor data and/or image data to detect the user input and provide an accurate representation of the user 702's motion. While four different input devices are shown (e.g., a wrist-wearable device 726, an MR device 732, an HIPD 742, and a smart textile-based garment 738) each one of these input devices entirely on its own can provide inputs for fully interacting with the MR environment. For example, the wrist-wearable device can provide sufficient inputs on its own for interacting with the MR environment. In some embodiments, if multiple input devices are used (e.g., a wrist-wearable device and the smart textile-based garment 738) sensor fusion can be utilized to ensure inputs are correct. While multiple input devices are described, it is understood that other input devices can be used in conjunction or on their own instead, such as but not limited to external motion-tracking cameras, other wearable devices fitted to different parts of a user, apparatuses that allow for a user to experience walking in an MR environment while remaining substantially stationary in the physical environment, etc.

As described above, the data captured by each device is used to improve the user's experience within the MR environment. Although not shown, the smart textile-based garments 738 can be used in conjunction with an MR device and/or an HIPD 742.

While some experiences are described as occurring on an AR device and other experiences are described as occurring on an MR device, one skilled in the art would appreciate that experiences can be ported over from an MR device to an AR device, and vice versa.

Some definitions of devices and components that can be included in some or all of the example devices discussed are defined here for ease of reference. A skilled artisan will appreciate that certain types of the components described may be more suitable for a particular set of devices, and less suitable for a different set of devices. But subsequent reference to the components defined here should be considered to be encompassed by the definitions provided.

In some embodiments example devices and systems, including electronic devices and systems, will be discussed. Such example devices and systems are not intended to be limiting, and one of skill in the art will understand that alternative devices and systems to the example devices and systems described herein may be used to perform the operations and construct the systems and devices that are described herein.

As described herein, an electronic device is a device that uses electrical energy to perform a specific function. It can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices, and/or a subset of components of one or more electronic devices and facilitates communication, and/or data processing and/or data transfer between the respective electronic devices and/or electronic components.

The foregoing descriptions of FIGS. 7A-7C-2 provided above are intended to augment the description provided in reference to FIGS. 1A-6. While terms in the following description may not be identical to terms used in the foregoing description, a person having ordinary skill in the art would understand these terms to have the same meaning.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt in or opt out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A system, comprising:
one or more processors communicatively coupled with:
a wearable device, and
an artificial intelligence (AI) agent; and
memory including executable instructions that, when executed by the one or more processors, cause the one or more processors to perform:
in response to a user request, received via the wearable device worn by a user, to perform a web-based action, invoking the AI agent,
causing the AI agent to traverse webpages based on the user request, wherein the AI agent obtains, for each webpage of the webpages, information for performing the web-based action,
generating, by the AI agent, an AI response based on the information, wherein the AI response includes one or more recommendations for performing the web-based action,
presenting, at the wearable device, the AI response, and
in response to user selection, via the wearable device, of a recommendation included in the AI response, causing the AI agent to perform the web-based action based on the recommendation.

2. The system of claim 1, wherein:
the one or more processors are communicatively coupled with one or more of:
one or more sensors, and
one or more imaging devices; and
the user request includes sensor data captured by the one or more sensors and image data captured by the one or more imaging devices.

3. The system of claim 1, wherein the instructions, when executed by the one or more processors, further cause the one or more processors to perform:
providing the AI agent user data including one or more of messages, calendar data, contact data, and application data; and
wherein the AI response is further based on the user data.

4. The system of claim 1, wherein the web-based action includes one or more of scheduling a reservation, locating a restaurant, locating an event, and organizing an event.

5. The system of claim 1, wherein causing the AI agent to perform the web-based action includes causing the AI agent to interact with at least one webpage associated with the recommendation.

6. The system of claim 1, wherein the instructions, when executed by the one or more processors, further cause the one or more processors to perform:
while one or more of causing the AI agent to traverse the webpages and generating the AI response, presenting an AI agent symbol representing progress in generating the AI response.

7. The system of claim 1, wherein causing the AI agent to perform the web-based action based on the recommendation includes providing the recommendation to at least one other electronic device associated with another user.

8. A method, comprising:
in response to a user request, received via a wearable device worn by a user, to perform a web-based action, invoking an artificial intelligence (AI) agent;
causing the AI agent to traverse webpages based on the user request, wherein the AI agent obtains, for each webpage of the webpages, information for performing the web-based action;
generating, by the AI agent, an AI response based on the information, wherein the AI response includes one or more recommendations for performing the web-based action;
presenting, at the wearable device, the AI response; and
in response to user selection, via the wearable device, of a recommendation included in the AI response, causing the AI agent to perform the web-based action based on the recommendation.

9. The method of claim 8, wherein:
the user request includes one or more of sensor data captured by one or more sensors and image data captured by the one or more imaging devices.

10. The method of claim 8, further comprising:
providing the AI agent user data including one or more of messages, calendar data, contact data, and application data; and
wherein the AI response is further based on the user data.

11. The method of claim 8, wherein the web-based action includes one or more of scheduling a reservation, locating a restaurant, locating an event, and organizing an event.

12. The method of claim 8, wherein causing the AI agent to perform the web-based action includes causing the AI agent to interact with at least one webpage associated with the recommendation.

13. The method of claim 8, further comprising:
while one or more of causing the AI agent to traverse the webpages and generating the AI response, presenting an AI agent symbol representing progress in generating the AI response.

14. The method of claim 8, wherein causing the AI agent to perform the web-based action based on the recommendation includes providing the recommendation to at least one other electronic device associated with another user.

15. A non-transitory computer-readable storage medium including instructions that, when executed by one or more processors of a system communicatively coupled with a wearable device and an artificial intelligence (AI) agent, cause the one or more processors to perform:
in response to a user request, received via a wearable device worn by a user, to perform a web-based action, invoking the AI agent;
causing the AI agent to traverse webpages based on the user request, wherein the AI agent obtains, for each webpage of the webpages, information for performing the web-based action;
generating, by the AI agent, an AI response based on the information, wherein the AI response includes one or more recommendations for performing the web-based action;
presenting, at the wearable device, the AI response; and
in response to user selection, via the wearable device, of a recommendation included in the AI response, causing the AI agent to perform the web-based action based on the recommendation.

16. The non-transitory computer-readable storage medium of claim 15, wherein:
the system is communicatively coupled with one or more of:
one or more sensors, and
one or more imaging devices; and
the user request includes one or more of sensor data captured by one or more sensors and image data captured by the one or more imaging devices.

17. The non-transitory computer-readable storage medium of claim 15, wherein the instructions, when executed by the one or more processors, further cause the system to perform:
providing the AI agent user data including one or more of messages, calendar data, contact data, and application data; and
wherein the AI response is further based on the user data.

18. The non-transitory computer-readable storage medium of claim 15, wherein the web-based action includes one or more of scheduling a reservation, locating a restaurant, locating an event, and organizing an event.

19. The non-transitory computer-readable storage medium of claim 15, wherein causing the AI agent to perform the web-based action includes causing the AI agent to interact with at least one webpage associated with the recommendation.

20. The non-transitory computer-readable storage medium of claim 15, wherein the instructions, when executed by the one or more processors, further cause the system to perform:
while one or more of causing the AI agent to traverse the webpages and generating the AI response, presenting an AI agent symbol representing progress in generating the AI response.
